(19) United States
(12) Patent Application Publication
(10) Pub. No.: US 2024/0102047 A1
(43) Pub. Date: Mar. 28, 2024

(54) An oncolytic virus vector coding for variant interleukin-2 (vIL-2) polypeptide

(71) Applicant: TILT Biotherapeutics Oy, Helsinki (FI)

(72) Inventors: Sadia Zafar, Helsinki (FI); Dafne Quixabeira, Helsinki (FI); Riikka Havunen, Helsinki (FI); Akseli Hemminki, Helsinki (FI)

(21) Appl. No.: 17/767,945

(22) PCT Filed: Oct. 12, 2020
(86) PCT No.: PCT/FI2020/050673

§ 371 Date: Apr. 11, 2022

(30) Foreign Application Priority Data
Oct. 11, 2019 (FI) 20195876

(51) Int. Cl. C12N 15/86; C07K 14/525; C07K 14/55

(52) U.S. Cl. CPC C12N 15/86; C07K 14/525; C07K 14/55; A61K 38/00; C12N 2710/10032

(57) ABSTRACT

The present invention provides an oncolytic adenoviral vector comprising a nucleic acid sequence encoding a variant interleukin 2 (vIL-2) polypeptide as a transgene. The present invention also provides a pharmaceutical composition comprising said oncolytic vector and at least one of the following: physiologically acceptable carriers, buffers, excipients, adjuvants, additives, antiseptics, preservatives, filling, stabilising and/or thickening agents. A particular aim of the present invention is to provide said oncolytic viral vector or pharmaceutical composition for use in the treatment of cancer or tumor, preferably a solid tumor.

### FIELD OF THE INVENTION

The present invention relates to the fields of life sciences and medicine. Specifically, the invention relates to cancer therapies of humans. More specifically, the present invention relates to an oncolytic viral vector comprising a nucleic acid sequence encoding a variant interleukin-2 (vIL-2) polypeptide.

### BACKGROUND OF THE INVENTION

The immunostimulatory cytokine interleukin-2 (IL-2) belongs to the family of γ-chain cytokines. It is a growth factor of leukocytes, such as T cells and natural killer (NK) cells. IL-2 is produced primarily by activated CD4+ and CD8+ T lymphocytes and has various immunological effects, such as inducing T cell proliferation and activation, potentiating B cell growth and activating monocytes and natural killer cells. IL-2 has been investigated as a therapeutic agent for a wide range of immune disorders, but adverse effects related to systemic administration of high IL-2 doses have limited its clinical application. IL-2 signals by binding to its receptor, which consist of three subunits: IL-2Rγ (or CD132), IL-2Rβ (or CD122) and IL-2Rα (or CD25). Both CD8+ and CD4+ T cells, including regulatory T cells (CD4+Foxp3+; Tregs), express the trimeric form constitutively. The dimeric intermediate form of IL-2 receptor, consisting of IL-2Rγ and IL-2Rβ subunits, is expressed on NK cells and resting CD8+ and CD4+ T cells.

The ability of IL-2 to expand and activate CD8+ effector cells encouraged its application in the treatment of renal cell carcinoma and melanoma. As a downside, IL-2 also plays a central role in the expansion and maintenance of immunosuppressive regulatory cells, mainly Tregs. Although IL-2 therapy has shown long-lasting responses in some patients, systemic delivery has demonstrated limitations in several clinical trials. High-dose IL-2 is needed for the effective treatment, causing liver, heart, and lung problems, while the antitumor efficacy is compromised through Treg induction.

In the prior art, Levin et al., 2012, eliminated the functional requirement of IL-2 for CD25 expression by engineering an IL-2 ‘superkine’ (also called super-2) with increased binding affinity for IL-2Rβ. Compared to IL-2, the IL-2 superkine induced superior expansion of cytotoxic T cells, leading to improved antitumour responses in vivo, and elicited proportionally less expansion of T regulatory cells and reduced pulmonary oedema.

U.S. Pat. No. 9,428,567 discloses human interleukin-2 (hIL-2) variants having an equilibrium dissociation constant for the IL-2Rβ subunit which is less than that of wild-type human IL-2. The variants may also exhibit reduced binding to the IL-2Rα or IL-2Rγ subunit relative to wild type IL-2.

Given the ability of IL-2 to stimulate T-cells, a virus coding for IL-2 would have potential for enhancing T-cell therapies (Itzhaki et al., 2013; Schwartz et al., 2002). T-cell therapies include tumor-infiltrating lymphocytes (TILs), receptor-modified T cells (TCR) and chimeric antigen receptor T-cells (CAR-T). T cells are extracted from patient's blood or tumor, activated and/or modified in laboratory, expanded, and given back to the patient as a therapeutic regimen (Tähtinen et al., 2016). However, because the highly immunosuppressive tumor microenvironment renders adoptively transferred T cells hypofunctional, T-cell infusion requires pre- and postconditioning with chemotherapeutics and high-dose systemic IL-2, respectively, which both cause severe toxicities (Schwartz, Stover et al. 2002, Itzhaki, Levy et al. 2013).

After years of development, the oncolytic viruses are currently starting to be used as cancer therapeutics. Although there have been some discoveries relating to the mechanisms of action and factors that influence the efficacy of the viruses, there is still a need to identify pathways that determine the overall response to virotherapy. In clinical trials, oncolytic viruses have demonstrated a favorable safety profile and promising efficacy.

WO2014170389 relates to oncolytic adenoviral vectors alone or together with therapeutic compositions for therapeutic uses and therapeutic methods for cancer. For instance, a separate administration of adoptive cell therapeutic composition and oncolytic adenoviral vectors is disclosed. Adoptive cell therapies (ACT) are a potent approach for treating cancer but also for treating other diseases such as infections and graft versus host disease. Adoptive cell transfer is the passive transfer of ex vivo grown cells, most commonly immune-derived cells, into a host with the goal of transferring the immunologic functionality and characteristics of the transplant. WO2014170389 also discloses nucleic acid sequences of oncolytic adenoviral vectors.

WO2016146894 discloses an oncolytic adenoviral vector encoding a bispecific monoclonal antibody.

US2019062395 discloses a modified oncolytic vaccinia virus vector comprising a transgene encoding an IL-2 variant.

There is still room for improvement in the responses to oncolytic viral treatments, especially in patients with a significant metastasis burden. Further characterization of pathways related to the activity of oncolytic viruses could reveal potential targets for improving the efficacy of virotherapy. Therefore, the efficacy of oncolytic viral vectors, either alone or together with other therapies, can still be improved. The present invention provides efficient tools and methods for cancer therapeutics by utilizing specific viral vectors, e.g. with adoptive cell therapies.

### SUMMARY OF THE INVENTION

The aim of this invention is to overcome the limitations seen in the use of IL-2 therapy, chiefly, the stimulation of immunosuppressive Tregs. We designed an oncolytic adenoviral vector expressing a variant IL-2 (vIL-2) polypeptide as a transgene. The vIL-2 gene has point mutations in the natural IL-2 gene to abolish its binding to CD25 (receptor subunit a). The vIL2 thus expressed is therefore unable to stimulate Treg cells, resulting in a preferred expansion of cytotoxic T cells. In this construct, virus replication is restricted to cancer cells and transgene (vIL-2) expression is linked to the virus replication. Thus, vIL-2 is only expressed where it is needed: in the tumor microenvironment. Virus replication within the cancer cells causes danger signaling and spreading of tumor-associated antigens, which facilitates recognition of the cancer cells by the immune system for killing of the cells. Moreover, expression of immunostimulatory cytokine further boosts this effect.

Accordingly, an object of the present invention is to provide simple methods and tools for overcoming the problems of inefficient, unsafe and unpredict-able cancer therapies. In one embodiment, the invention provides novel methods and means for cell therapy. The objects of the invention are achieved by specific viral vectors, methods and arrangements, which are characterized by what is stated in the independent claims. The specific embodiments of the invention are disclosed in the dependent claims.

Specifically, the present invention provides an oncolytic adenoviral vector comprising a nucleic acid sequence encoding a variant interleukin 2 (vIL-2) polypeptide as a transgene. The present invention also provides a pharmaceutical composition comprising said oncolytic vector and at least one of the following: physiologically acceptable carriers, buffers, excipients, adjuvants, additives, antiseptics, preservatives, filling, stabilising and/or thickening agents. A particular aim of the present invention is to provide said oncolytic viral vector or pharmaceutical composition for use in the treatment of cancer or tumor, preferably a solid tumor.

### BRIEF DESCRIPTION OF THE DRAWINGS

FIG. 1. Variant IL-2 (vIL-2) has more beneficial effects on immune cell population proliferation than the conventional IL-2. Recombinant human (rh) vIL-2 induces considerable increase in A) CD3+CD8+ T cell and B) CD3-T CD56+NK cell populations as compared with conventional rhIL-2. C) rhIL-2 induces the proliferation of CD3+T CD4+ T cell population more than (rh)vIL-2. After 3 days, the (rh)vIL-2 was more potent in inducing the proliferation of CD8+ effector T cells and NK cells than rhIL-2, whereas the levels of CD4+ T cells (including Tregs) remained lower with the variant. Data are presented as mean+standard error of means (SEM). **p<0.01.

FIG. 2. The effects of vIL-2 on Effector and Exhausted T Cells. A) CD8/CD4+ T cell ratio of CD3+ T cell parent population that was pre-activated and cultured with rhIL-2, (rh)vIL-2, or media only. Alternatively, the pre-activated T cells were cultured with rhIL-2, (rh)vIL-2, or media only in the presence of non-infected (B) or infected (C) cancer cells. rhIL-2 and (rh)vIL-2 had similar effects on CD8/CD4 cell ratios in the presence and absence of cancer cells (A and B). However, when the cancer cells were infected with an oncolytic virus, rhvIL-2 induced a trend towards CD8+CD27-CD62L-CD45RO+ cell dominance over CD4+ cells (C). Mean is shown. Virus: Ad5/3-E2F-d24; CC: Cancer cells.

FIG. 3. The effects of vIL-2 on Central Memory T Cells. A) CD8/CD4+ T cell ratio of CD3+ T cell parent population was pre-activated and cultured with rhIL-2, (rh)vIL-2, or media only. Alternatively, the pre-activated T cells were cultured with rhIL-2, (rh)vIL-2, or media only in the presence of non-infected (B) or infected (C) cancer cells. We did not find any difference in CD8/CD4 Tcm ratios between rhIL-2 and (rh)vIL-2 in the absence of cancer cells (A). In the presence of tumor cells, we first observed a decrease in the ratio on day 2, followed by an increase on day 4. Again, the (rh)vIL-2 induced higher CD8 to CD4 ratio in the Tcm population than the conventional rhIL-2 (B). In the presence of backbone virus, CD8/CD4 Tcm cells were higher in the group with (rh)vIL-2 on day 2, and remains steady till day 4, the ratio of these cells in other groups increased as compared to the (rh)vIL-2 group (C). This indicates that (rh)vIL-2 influence Tcm compartment in a similar way as rhIL-2. Mean is shown. Virus: Ad5/3-E2F-d24, CC: Cancer cells.

FIG. 4. The effects of vIL-2 on Effector Memory T Cells. A) CD8/CD4+ T cell ratio of CD3+ T cell parent population was pre-activated and cultured with rhIL-2, (rh)vIL-2, or media only. Alternatively, the pre-activated T cells were cultured with rhIL-2, (rh)vIL-2, or media only in the presence of non-infected (B) or infected (C) cancer cells. We did not observe differences in CD8/CD4 Tem ratio between rhIL-2 and (rh)vIL-2, if cancer cells were not present (A). With cancer cells, rhIL-2 and (rh)vIL-2 induce high ratio of CD8/CD4 Tem by day 4 (B). When the cancer cells were infected, we observed a trend towards high CD8/CD4 Tem ratio in (rh)vIL-2 group on day 4 (C). Mean is shown. Virus: Ad5/3-E2F-d24, CC: Cancer cells.

FIG. 5. The constructed virus is oncolytic and has a backbone from a common cold virus, adenovirus serotype 5. A) A schematic presentation of chimeric 5/3 oncolytic adenovirus with E2F promoter; 24-base-pair deletion in E1A; disabling deletion of E1B; human vIL-2 transgene inserted in the E3 region; and an Ad3 serotype knob in the Ad5 fiber. B) The virus has oncolytic potency ex vivo. BB indicates the backbone virus without transgenes C) Virus-infected cells secrete transgene products into the growth medium. There were no major differences between viruses' cell killing ability between Ad5/3-E2F-d24-IL-2 and Ad5/3-E2F-d24-vIL-2, thus indicating that presence of vIL-2 transgene does not reduce the oncolytic potency of the virus (B). Additionally, cells infected with Ad5/3-E2F-d24-vIL-2 were able to secrete the cytokine (C).

FIG. 6. Oncolytic adenovirus Ad5/3-E2F-d24-vIL-2 induces CD8+ effector cell dominance and does not induce Treg differentiation. A) Unlike the virus coding for conventional IL-2, Ad5/3-E2F-d24-vIL-2 induces the presence of activated effector T cells (CD3+ CD8+ CD25+ CD69+) over the activated CD4+ T cells. B) The virus coding for conventional IL-2 stimulates the differentiation of immunosuppressive Tregs, unlike Ad5/3-E2F-d24-vIL-2. The CD8/CD4 ratio of CD25+ CD69+ activated effector T cells was significantly higher in the group treated with Ad5/3-E2F-d24-vIL-2 on day 3 and day 6, than when treated with the virus expressing conventional IL-2 (A). Ad5/3-E2F-d24-vIL-2 did not induce Treg differentiation like Ad5/3-E2F-d24-IL-2 (B). Data are presented as mean+SEM. ****p<0.0001; **p=0.01. Ad5/3-vIL-2: Ad5/3-E2F-d24-vIL2; Ad5/3-IL-2: Ad5/3-E2F-d24-IL2; Ad5/3: Ad5/3-E2F-d24; PBMCs: Human peripheral blood mononuclear cells.

FIG. 7. Ad5/3-E2F-d24-vIL2 enhanced antitumor efficacy and overall survival in hamsters: 2*10⁶ HapT1 tumors were implanted subcutaneously in Syrian hamsters. (A-D) Individual tumor growth till day 16 for hamsters treated with 1*10⁹ VP of Ad5/3-E2F-d24-IL-2, Ad5/3-E2F-d24-vIL-2 or unarmed control virus Ad5/3-E2F-d24 and mock received PBS on day 1, 4, 8 and 13. Ad5/3-E2F-d24-vIL-2 showed better tumor control as compared with other groups. (E) Hamsters with established HapT1 tumors were treated with different adenoviruses on days 1, 4, 8, 13. Starting from day 18, groups received six additional rounds of treatment every 5 days. Ad5/3-E2F-d24-vIL2 significantly reduced tumor growth as compared with other groups, including Ad5/3-E2F-d24-IL-2. Hamsters were considered as cured when tumors were no longer visible. Normalized median tumor volume and SEM. ***, P<0.001; *, P<0.05. (F) Percentage of cured tumors by day 30. (G) Overall survival and statistical significances.

FIG. 8: Induction of Tumor-Specific immunological memory after treatment with cytokine armed adenovirus: All hamsters, which were cured of HapT1 tumors were rechallenged with (A) HapT1 (the same tumor) and with (B) DDT1-MF2 (a different tumor), implanted on the upper back of hamsters. Previous treatment with cytokine-armed adenovirus appeared to reduce growth of HapT1 cells following rechallenge. Most importantly, vIL-2 armed adenovirus was able to induce complete tumor rejection in 40% (2 out of 5) of the animals following HapT1 rechallenge.

FIG. 9. Variant IL-2-virus treatment achieves substantial tumor reduction and moderate infiltration levels of CD4+ and CD8+at the tumor microenvironment. HapT1-bearing hamsters were treated 4 times with intratumoral injections of PBS (Mock), or Ad5/3-E2F-d24, or Ad5/3-E2F-d24-IL-2, or Ad5/3-E2F-d24-vIL-2. Tumors were collected from hamsters at day 16 (after 4 treatments) for detection of immune cells through flow cytometry. (A) Tumor volumes at day 0 and 16. (B) Frequency of CD4+ cells, (C) Frequency of CD8+ cells. Data is presented as mean+SEM. *p<0.05

FIG. 10. Variant IL-2-virus treatment achieves high-levels of IL-2 at the tumor microenvironment. Tumors were collected from hamsters at day 16 (after 4 treatments) for relative mRNA quantification through RT-qPCR. Intratumoral relative mRNA expression levels of hamster IL-2 (lower bars), human IL-2 and IL-2 variant (upper bars). Data is presented as mean+SEM. *p<0.05,****p<0.0001

FIG. 11. Overall mRNA expression profile of virus-treated animals. Tumors were collected from hamsters at day 16 and mRNA expression profile was determined through Nanostring. (A) mRNA expression profile of Ad5/3-E2F-d24, (B) mRNA expression profile of Ad5/3-E2F-d24-IL-2 (C) mRNA expression profile of Ad5/3-E2F-d24-IL-2 variant. Names are indicated for genes which were statistically significant different (adjusted p value<0.05) expression compared to reference group (−1>log 2 fold change>1).

FIG. 12. mRNA expression levels of T-cell receptor signaling and cytotoxic compounds in tumors treated with oncolytic adenovirus coding for wild-type human IL-2 or variant IL-2. Tumors were collected from hamsters at day 16 and mRNA expression levels were determined through Nanostring. (A) mRNA count for genes related to T-cell receptor (TCR)-complex and signaling, (B) mRNA count for genes related to cytotoxic compounds, (C) Pearson's correlation between variant IL-2 mRNA relative expression and GZMK or SAP1 mRNA counts, or between both the latter genes. Data is presented as mean+SEM from genes which were statistically different from the reference group (Mock). ns—non-significant

FIG. 13. mRNA expression levels of anti-inflammatory and pro-inflammatory signal genes in tumors treated with oncolytic adenovirus coding for human IL-2 or variant IL-2. Tumors were collected from hamsters at day 16 and mRNA expression levels were determined through Nanostring. (A) mRNA count for genes which are related with co-stimulatory and co-inhibitory molecules. (B) mRNA count for genes which are related with antigen-presenting cells and suppressive myeloid cells. (C) mRNA count for genes which are related with signals associated with anti-inflammatory and pro-inflammatory signals. Data is presented as mean+SEM from genes which were statistically different from the reference group (Mock). *p<0.05,**p<0.01

### DETAILED DESCRIPTION OF THE INVENTION

### Interleukin 2 (IL-2) and Variants Thereof

As used herein, “IL-2” means wild-type IL-2, whether native or recombinant. Mature human IL-2 occurs as a 133 amino acid sequence (without the signal peptide, consisting of an additional 20 N-terminal amino acids). The amino acid sequence of human IL-2 (SEQ ID NO: 1) is found in Genbank under accession number NP000577.2. The amino acid sequence of mature human IL-2 is depicted in SEQ ID NO: 2.

As used herein, “IL-2 variant”, “variant IL-2”, “vIL2” or “vIL-2” means a polypeptide or a nucleic acid (i.e. a gene) encoding said polypeptide, wherein specific substitutions to the interleukin-2 polypeptide have been made. The term “polypeptide” refers herein to any chain of amino acid residues, regardless of its length or post-translational modification (e.g., glycosylation or phosphorylation). The variant IL-2 polypeptides can also be characterized by amino acid insertions, deletions, substitutions and modifications at one or more sites in or at the other residues of the native IL-2 polypeptide chain. In accordance with this disclosure any such insertions, deletions, substitutions and modifications result in a variant IL-2 that preferably exhibits reduced binding to receptor subunit IL-2α but retains or improves the IL-2Rβ binding activity. Exemplary variants can include substitutions of 1, 2, 3, 4, 5, 6, 7, 8, 9, 10 or more amino acids. Variants may also include conservative modifications and substitutions at other positions of IL-2 (i.e., those that have a minimal effect on the activity or secondary or tertiary structure of the variant).

An exemplary variant IL-2 polypeptide includes an amino acid sequence that is at least about 80% identical to SEQ ID NO:2 which binds the IL-2Rα with an affinity that is lower than the affinity with which the polypeptide represented by SEQ ID NO: 2 binds the IL-2Rα. Exemplary variant IL-2 polypeptides can be at least about 50%, at least about 65%, at least about 70%, at least about 80%, at least about 85%, at least about 87%, at least about 90%, at least about 95%, at least about 97%, at least about 98%, or at least about 99% identical to wild-type IL-2. The variant polypeptide can comprise a change in the number or content of amino acid residues. For example, the variant IL-2 can have a greater or a lesser number of amino acid residues than wild-type IL-2. Alternatively, or in addition, an exemplary variant polypeptide can contain a substitution of one or more amino acid residues that are present in the wild-type IL-2. In various embodiments, the variant IL-2 polypeptide can differ from wild-type IL-2 by the addition, deletion, or substitution of a single amino acid residue, for example, a substitution of the residue at position 80 of SEQ ID NO:2. Similarly, exemplary variant polypeptides can differ from wild-type by a substitution of two or more amino acid residues, for example, the residues at positions 24, 45, 65, 72, 74, 80, 81, 85, 86, 89, 92, 93, 109 and 117 of SEQ ID NO:2. For example, the mutation can be selected from the group of consisting of: I24V, Y45A P65H, L72G, Q74R, Q74H, Q74N, Q74S, L80F, L80V, R81I, R81T, R81D, L85V, I86V, I89V, I92F, V93I, D109L, F117A. Preferably, the variant polypeptide comprises the substitutions L80F, R81D, L85V, I86V and I92F.

In another embodiment, variant IL-2 polypeptides can also be prepared as fusion or chimeric polypeptides that include a variant IL-2 polypeptide and another heterologous polypeptide. A chimeric polypeptide including a variant IL-2 and an antibody or antigen-binding portion thereof can be generated. The antibody or antigen-binding component of the chimeric protein can serve as a targeting moiety. For example, it can be used to localize the chimeric protein to a particular subset of cells or target molecule.

The present invention is particularly directed to a design of an oncolytic viral vector comprising nucleic acid sequence encoding any of the above-mentioned variant IL-2 polypeptides as a transgene.

### Viral Vectors

Oncolytic viral vectors are therapeutically useful anticancer viruses that can selectively infect and destroy cancer cells. Most current oncolytic viruses are adapted or engineered for tumour selectivity, although there are viruses, such as reovirus and Mumps virus, having natural preference for cancer cells. Many engineered oncolytic viral vectors take advantage of tumor-specific promoter elements making them replication competent only in cancer cells. Surface markers expressed selectively by cancer cells can also be targeted by using them as receptors for virus entry. A number of viruses including adenovirus, reovirus, measles, herpes simplex, Newcastle disease virus and vaccinia have now been clinically tested as oncolytic agents.

Preferably, the oncolytic vector used in the present invention is an adenoviral vector suitable for treating a human or animal. As used herein “an oncolytic adenoviral vector” refers to an adenoviral vector capable of infecting and killing cancer cells by selective replication in tumor versus normal cells.

In one embodiment of the invention, the adenoviral vectors are vectors of human viruses. In one embodiment the adenoviral vectors are selected from the group consisting of Ad5, Ad3 and Ad5/3 vectors. As used herein, expression “adenovirus serotype 5 (Ad5) nucleic acid backbone” refers to the genome of Ad5. Similarly, “adenovirus serotype 3 (Ad3) nucleic acid backbone” refers to the genome of Ad3. “Ad5/3 vector” refers to a chimeric vector comprising or having parts of both Ad5 and Ad3 vectors. In a specific embodiment a backbone of the adenoviral vector is an adenovirus serotype 5 (Ad5) or serotype 3 (Ad3) nucleic acid backbone with specific mutations. E.g. fiber areas of the vector can be modified. In one embodiment the backbone is Ad5 nucleic acid backbone further comprising an Ad3 fiber knob. In other words the construct has the fiber knob from Ad3 while the remainder or the most of the remainder of the genome is from Ad5 (see, e.g., WO2014170389).

The adenoviral vectors may be modified in any way known in the art, e.g. by deleting, inserting, mutating or modifying any viral areas. The vectors are made tumor specific with regard to replication. For example, the adenoviral vector may comprise modifications in E1, E3 and/or E4 such as insertion of tumor specific promoters (e.g. to drive E1), deletions of areas (e.g. the constant region 2 of E1 as used in “424”, E3/gp19k, E3/6.7k) and insertion of a transgene or transgenes.

In a specific embodiment, the E1B 19K gene (SEQ ID NO:3), generally known to support replication of adenoviral vectors, has a disabling deletion dE1B 19K (SEQ ID NO:4) in the present vectors. Deletion of E1B 19K is known to sensitize cancer cells to TNFalpha and thus it promotes apoptosis (White et al., 1992).
The sequence for wild-type E1B 19K gene is the following (the deletable region is underlined):

(SEQ ID NO: 3)
atggaggctt gggagtgttt ggaagatttt tctgctgtgc
gtaacttgct ggaacagagc tctaacagta cctcttggtt
ttggaggttt ctgtggggct catcccaggc aaagttagtc
tgcagaatta aggaggatta caagtgggaa tttgaagagc
ttttgaaatc ctgtggtgag ctgtttgatt ctttgaatct
gggtcaccag gcgcttttcc aagagaaggt catcaagact
ttggattttt ccacaccggg gcgcgctgcg gctgctgttg
cttttttgag ttttataaag gataaatgga gcgaagaaac
ccatctgagc ggggggtacc tgctggattt tctggccatg
catctgtgga gagcggttgt gagacacaag aatcgcctgc
tactgttgtc ttccgtccgc ccggcgataa taccgacgga
ggagcagcag cagcagcagg aggaagccag gcggcggcgg
caggagcaga gcccatggaa cccgagagcc ggcctggacc
ctcgggaatg a

Accordingly, in an embodiment, the sequence for dE1B 19K in the present viral vectors is

(SEQ ID NO: 4)
atggaggctt gggagtgttt ggaagatttt tctgctgtgc
gtaacttgct ggaacagctg ggtcaccagg cgcttttcca
agagaaggtc atcaagactt tggatttttc cacaccgggg
cgcgctgcgg ctgctgttgc ttttttgagt tttataaagg
ataaatggag cgaagaaacc catctgagcg gggggtacct
gctggatttt ctggccatgc atctgtggag agcggttgtg
agacacaaga atcgcctgct actgttgtct tccgtccgcc
cggcgataat accgacggag gagcagcagc agcagcagga
ggaagccagg cggcggcggc aggagcagag cccatggaac
ccgagagccg gcctggaccc tcgggaatga

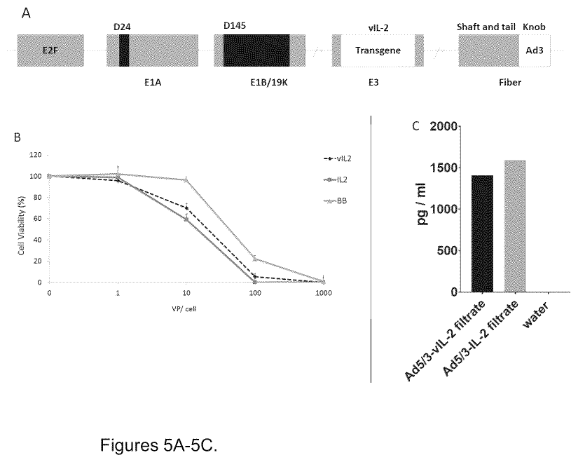

One approach for generation of a tumor specific oncolytic adenovirus is engineering a 24 base pair (bp) deletion (“Δ24” or “d24”) affecting the constant region 2 (CR2) of E1. In wild type adenovirus CR2 is responsible for binding the cellular Rb tumor suppressor/cell cycle regulator protein for induction of the synthesis (S) phase i.e. DNA synthesis or replication phase. The interaction between pRb and E1A requires amino acids 121 to 127 of the E1A protein conserved region. The vector may comprise a deletion of nucleotides corresponding to amino acids 122-129 of the vector according to Heise C. et al. (2000, Nature Med 6, 1134-1139) and Fueyo J. et al. (2000, Oncogene 19(1):2-12). Viruses with the Δ24 are known to have a reduced ability to overcome the G1-S checkpoint and replicate efficiently only in cells where this interaction is not necessary, e.g. in tumor cells defective in the Rb-p16 pathway, which includes most if not all human tumors. In one embodiment of the invention the vector comprises a 24 bp deletion (“Δ24” or “d24”) in the Rb binding constant region 2 of adenoviral E1 (See FIG. 5).

It is also possible to replace E1A endogenous viral promoter for example by a tumor specific promoter. For instance, E2F1 (e.g. in Ad5 based vector) or hTERT (e.g. in Ad3 based vector) promoter can be utilized in the place of E1A endogenous viral promoter. The vector may comprise E2F1 promoter for tumor specific expression of E1A.

The E3 region is nonessential for viral replication ex vivo, but the E3 proteins have an important role in the regulation of host immune response i.e. in the inhibition of both innate and specific immune responses. In one embodiment of the invention the deletion of a nucleic acid sequence in the E3 region of the oncolytic adenoviral vector is a deletion of viral gp19k and 6.7k reading frames. The gp19k/6.7K deletion in E3 refers to a deletion of 965 base pairs from the adenoviral E3A region. In a resulting adenoviral construct, both gp19k and 6.7K genes are deleted (Kanerva A et al. 2005, Gene Therapy 12, 87-94). The gp19k gene product is known to bind and sequester major histocompatibility complex I (MHC1, known as HLA1 in humans) molecules in the endoplasmic reticulum, and to prevent the recognition of infected cells by cytotoxic T-lymphocytes. Since many tumors are deficient in HLA1/MHC1, deletion of gp19k increases tumor selectivity of viruses (virus is cleared faster than wild type virus from normal cells but there is no difference in tumor cells). 6.7K proteins are expressed on cellular surfaces and they take part in downregulating TNF-related apoptosis inducing ligand (TRAIL) receptor 2.

In one embodiment of the invention, the transgene, i.e. a gene encoding variant interleukin 2 (vIL2), is placed into a gp19k/6.7k deleted E3 region, under the E3 promoter. This restricts transgene expression to tumor cells that allow replication of the virus and subsequent activation of the E3 promoter. In a specific embodiment a nucleic acid sequence encoding variant interleukin 2 is inserted into the place of the deleted nucleic acid sequence of viral gp19k and 6.7k reading frames. In another embodiment of the invention E3 gp19k/6.7k is kept in the vector but one or many other E3 areas have been deleted (e.g. E3 9-kDa, E3 10.2 kDa, E3 15.2 kDa and/or E3 15.3 kDa).

E3 promoter may be any exogenous (e.g. CMV or E2F promoter) or endogenous promoter known in the art, specifically the endogenous E3 promoter. Although the E3 promoter is chiefly activated by replication, some expression occurs when E1 is expressed. As the selectivity of 424 type viruses occurs post E1 expression (when E1 is unable to bind Rb), these viruses do express E1 also in transduced normal cells. Thus, it is of critical importance to regulate also E1 expression to restrict E3 promoter mediated transgene expression to tumor cells.

Specific embodiments of the invention include oncolytic adenoviral vectors (e.g. Ad5 or Ad3 vectors) whose replication is restricted to the p16/Rb pathway by dual selectivity devices: an E2F (e.g. E2F1) tumor specific promoter placed in front of the adenoviral E1A gene which has been mutated in constant region 2, so that the resulting E1A protein is unable to bind Rb in cells. Furthermore, the fiber is modified by 5/3 chimerism to allow efficient entry into tumor cell.

In a specific embodiment of the invention the oncolytic adenoviral vector comprises:
1) a 24 bp deletion (Δ24) in the Rb binding constant region 2 of adenoviral E1;
2) a nucleic acid sequence deletion of viral gp19k and 6.7k reading frames; and
3) a nucleic acid sequence encoding a variant interleukin 2 (vIL2) transgene in the place of the deleted nucleic acid sequence as defined in point 2).

In the Experimental Section below, we constructed and characterized an oncolytic adenovirus based on Ad5/3-E2F-d24 backbone and armed it with vIL2. The virus has an E2F promoter and a 24-base pair deletion in the E1A constant region 2 (“D24”) to enable its replication only in retinoblastoma/p16 pathway-defective cells, which is one of the common features for all cancer cells. E1B region is deleted to induce cancer cell apoptosis (dE1B 19K). Moreover, to improve its ability to transduce cancer cells and enhance its antitumor efficacy, the virus features fiber knob from serotype 3, while the rest of the genome derives from serotype 5. Most importantly, Ad5/3 viruses have good safety profile in humans. Preferably, oncolytic virus armed with vIL-2 is used with concomitant T-cell therapy or checkpoint inhibitors, as a potential platform to safely and effectively treat currently incurable solid tumors. In particular, tumor types where Tregs play an important role are preferably treated.

In an embodiment, the present invention is directed to an oncolytic viral vector, preferably an oncolytic adenoviral vector, comprising a nucleic acid sequence encoding a variant interleukin 2 (vIL2) transgene.

In a preferred embodiment, the backbone of the oncolytic adenoviral vector is an adenovirus serotype 5 (Ad5) or serotype 3 (Ad3) nucleic acid backbone.

In a more preferred embodiment, said nucleic acid sequence encoding a variant interleukin 2 (vIL2) transgene is in the place of a deleted nucleic acid sequence in the E3 region of said oncolytic adenoviral vector. Most preferably, the deletion of a nucleic acid sequence in the E3 region is a deletion of viral gp19k and 6.7k reading frames.

In another preferred embodiment, the vector also comprises a 24 bp deletion (Δ24) in the adenoviral E1 sequence of said oncolytic adenoviral vector.

In another preferred embodiment, the vector also comprises a disabling deletion of E1B ((dE1B 19K).

In another preferred embodiment, the vector also comprises an Ad5/3 fiber knob.

In another preferred embodiment, the vector comprises nucleic acid sequence encoding a further transgene. More preferably, the further transgene is encoding a cytokine. In an embodiment, the cytokine is selected from the list consisting of: TNFalpha, interferon alpha, interferon beta, interferon gamma, complement C5a, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5 (=RANTES), CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2.

In a more preferred embodiment, the cytokine is TNFalpha.

The viral vectors utilized in the present inventions may also comprise other modifications than described above. Any additional components or modifications may optionally be used but are not obligatory for the present invention.

Insertion of exogenous elements may enhance effects of vectors in target cells. The use of exogenous tissue or tumor-specific promoters is common in recombinant vectors and they can also be utilized in the present invention.

### Adoptive Cell Therapy

One approach of the present invention is the development of a treatment for patients with cancer using the transfer of immune lymphocytes that are capable of reacting with and destroying the cancer. Isolated tumor-infiltrating lymphocytes are grown in culture to large numbers and infused into the patient. In the present invention oncolytic vectors encoding a variant interleukin 2 (vIL2) transgene may be utilized for increasing the effect of lymphocytes. As used herein “increasing the efficacy of adoptive cell therapy” refers to a situation, wherein the oncolytic vector of the invention is able to cause a stronger therapeutic effect in a subject when used together with an adoptive cell therapeutic composition compared to the therapeutic effect of the adoptive cell therapeutic composition alone. A specific embodiment of the invention is a method of treating cancer in a subject, wherein the method comprises administration of an oncolytic vector of the invention to a subject, said method further comprising administration of adoptive cell therapeutic composition to the subject. Adoptive cell therapeutic composition and the vectors of the invention are administered separately. Separate administrations of an adoptive cell therapeutic composition and adenoviral vectors may be preceded by myeloablating or non-myeloablating preconditioning chemotherapy and/or radiation. The adoptive cell therapy treatment is intended to reduce or eliminate cancer in the patient.

A specific embodiment of the invention relates to therapies with adenoviral vectors and an adoptive cell therapeutic composition, e.g. tumor-infiltrating lymphocytes, TCR modified lymphocytes or CAR modified lymphocytes. T-cell therapies in particular, but also any other adoptive therapies such as NK cell therapies or other cell therapies may be utilized in the present invention. Indeed, according to the present invention the adoptive cell therapeutic composition may comprise unmodified cells such as in TIL therapy or genetically modified cells. There are two common ways to achieve genetic targeting of T-cells to tumor specific targets. One is transfer of a T-cell receptor (TCR) with known specificity and with matched human leukocyte antigen (HLA, known as major histocompatibility complex in rodents) type. The other is modification of cells with artificial molecules such as chimeric antigen receptors (CAR). This approach is not dependent on HLA and is more flexible with regard to targeting molecules. For example, single chain antibodies can be used and CARs can also incorporate costimulatory domains. However, the targets of CAR cells need to be on the membrane of target cells, while TCR modifications can utilize intracellular targets.

As used herein “adoptive cell therapeutic composition” refers to any composition comprising cells suitable for adoptive cell transfer. In one embodiment of the invention the adoptive cell therapeutic composition comprises a cell type selected from a group consisting of a tumor-infiltrating lymphocyte (TIL), TCR (i.e. heterologous T-cell receptor) modified lymphocytes and CAR (i.e. chimeric antigen receptor) modified lymphocytes. In another embodiment of the invention, the adoptive cell therapeutic composition comprises a cell type selected from a group consisting of T-cells, CD8+ cells, CD4+ cells, NK-cells, dendritic cells, delta-gamma T-cells, regulatory T-cells and peripheral blood mononuclear cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one specific embodiment of the invention the adoptive cell therapeutic composition comprises T cells. As used herein “tumor-infiltrating lymphocytes” or TILs refer to white blood cells that have left the bloodstream and migrated into a tumor. Lymphocytes can be divided into three groups including B cells, T cells and natural killer cells. In another specific embodiment of the invention the adoptive cell therapeutic composition comprises T-cells which have been modified with target-specific chimeric antigen receptors or specifically selected T-cell receptors. As used herein “T-cells” refers to CD3+ cells, including CD4+ helper cells, CD8+ cytotoxic T-cells and γδ T cells.

In addition to suitable cells, adoptive cell therapeutic composition used in the present invention may comprise any other agents such as pharmaceutically acceptable carriers, buffers, excipients, adjuvants, additives, antiseptics, filling, stabilising and/or thickening agents, and/or any components normally found in corresponding products. Selection of suitable ingredients and appropriate manufacturing methods for formulating the compositions belongs to general knowledge of a person skilled in the art.

The adoptive cell therapeutic composition may be in any form, such as solid, semisolid or liquid form, suitable for administration. A formulation can be selected from a group consisting of, but not limited to, solutions, emulsions, suspensions, tablets, pellets and capsules. The compositions are not limited to a certain formulation; instead the composition can be formulated into any known pharmaceutically acceptable formulation. The pharmaceutical compositions may be produced by any conventional processes known in the art.

A combination of an oncolytic adenoviral vector of the invention and an adoptive cell therapeutic composition refers to use of an oncolytic adenoviral vector and an adoptive cell therapeutic composition together but as separate compositions. It is clear to a person skilled in the art that an oncolytic adenoviral vector of the present invention and an adoptive cell therapeutic composition are not used as one composition. Indeed, adenoviral vectors are not used for modifying the adoptive cells but for modifying the target tumor, so that the tumor is more amenable to the desired effects of the cellular transplant. In particular, the present invention enhances recruitment of the adoptive transplant to the tumor, and increases its activity there. In a specific embodiment of the invention oncolytic adenoviral vectors and an adoptive cell therapeutic composition of a combination are for simultaneous or sequential, in any order, administration to a subject.

### Checkpoint Inhibitor

Immune checkpoint proteins interact with specific ligands which send a signal into T cells that inhibits T-cell function. Cancer cells exploit this by driving high level expression of checkpoint proteins on their surface thereby suppressing the anti-cancer immune response.

A checkpoint inhibitor (also referred to as a CPI) as described herein is any compound capable of inhibiting the function of an immune checkpoint protein. Inhibition includes reduction of function as well as full blockade. In particular, the immune checkpoint protein is a human checkpoint protein. Thus, the immune checkpoint inhibitor is preferably an inhibitor of a human immune checkpoint.

Checkpoint proteins include, without limitation, CTLA-4, PD-1 (and its ligands PD-L1 and PD-L2), B7-H3, B7-H4, HVEM, TIM3, GAL9, LAG3, VISTA, KIR, BTLA, TIGIT and/or IDO. The pathways involving LAG3, BTLA, B7-H3, B7-H4, TIM3 and KIR are recognized in the art to constitute immune checkpoint pathways similar to the CTLA-4 and PD-1 dependent pathways. The immune checkpoint inhibitor can be an inhibitor of CTLA-4, PD-1 (and its ligands PD-L1 and PD-L2), B7-H3, B7-H4, HVEM, TIM3, GAL9, LAG3, VISTA, KIR, BTLA, TIGIT and/or IDO. In some embodiments, the immune checkpoint inhibitor is an inhibitor of PD-L1. Preferably, the immune checkpoint inhibitor is a monoclonal antibody that selectively binds to PD-L1, more preferably selected from the group consisting of: BMS-936559, LY3300054, atezolizumab, durvalumab and avelumab.

In some embodiments, the checkpoint inhibitor of the combination is an antibody. The term “antibody” as used herein encompasses naturally occurring and engineered antibodies as well as full length antibodies or functional fragments or analogs thereof that are capable of binding e.g. the target immune checkpoint or epitope (e.g. retaining the antigen-binding portion). The antibody for use according to the methods described herein may be from any origin including, without limitation, human, humanized, animal or chimeric and may be of any isotype with a preference for an IgG1 or IgG4 isotype and further may be glycosylated or non-glycosylated. The term antibody also includes bispecific or multispecific antibodies so long as the antibody(s) exhibit the binding specificity herein described.

### Cancer

The recombinant vectors of the present invention are replication competent in tumor cells. In one embodiment of the invention the vectors are replication competent in cells, which have defects in the Rb-pathway, specifically Rb-p16 pathway. These defective cells include all tumor cells in animals and humans. As used herein “defects in the Rb-pathway” refers to mutations and/or epigenetic changes in any genes or proteins of the pathway. Due to these defects, tumor cells overexpress E2F and thus, binding of Rb by E1 A CR2, that is normally needed for effective replication, is unnecessary. Further selectivity is mediated by the E2F promoter, which only activates in the presence of free E2F, as seen in Rb/p16 pathway defective cells. In the absence of free E2F, no transcription of E1 A occurs and the virus does not replicate. Inclusion of the E2F promoter is important to prevent expression of E1 A in normal tissues, which can cause toxicity both directly and indirectly through allowing transgene expression from the E3 promoter.

The present invention relates to approaches for treating cancer in a subject. In one embodiment of the invention, the subject is a human or a mammal, specifically a mammal or human patient, more specifically a human or a mammal suffering from cancer.

The approach can be used to treat any cancers or tumors, including both malignant and benign tumors, both primary tumors and metastases may be targets of the approach. In one embodiment of the invention the cancer features tumor-infiltrating lymphocytes. The tools of the present invention are particularly appealing for treatment of metastatic solid tumors featuring tumor-infiltrating lymphocytes. In another embodiment the T-cell graft has been modified by a tumor or tissue specific T-cell receptor of chimeric antigen receptor.

As used herein, the term “treatment” or “treating” refers to administration of at least oncolytic adenoviral vectors to a subject, preferably a mammal or human subject, for purposes which include not only complete cure but also prophylaxis, amelioration, or alleviation of disorders or symptoms related to a cancer or tumor. Therapeutic effect may be assessed by monitoring the symptoms of a patient, tumor markers in blood, or for example a size of a tumor or the length of survival of the patient

In another embodiment of the invention the cancer or tumor is selected from a group consisting of nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, esophagus cancer, gall bladder cancer, head and neck cancer, eye cancer, kidney cancer, Wilms' tumor, Kaposi's sarcoma, prostate cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer. Preferably, the cancer or tumor treated is selected from the group consisting of renal cancer, ovarian cancer, bladder cancer, prostate cancer, breast cancer, colorectal cancer, lung cancer (such as small-cell lung carcinoma, non-small-cell lung carcinoma and squamous non-small-cell lung carcinoma), gastric cancer, classical Hodgkin lymphoma, mesothelioma, and liver cancer. In a more preferred embodiment, the cancer or tumor type is head and neck cancer, most preferably human head and neck cancer.

Before classifying a human or animal patient as suitable for the therapy of the present invention, the clinician may examine a patient. Based on the results deviating from the normal and revealing a tumor or cancer, the clinician may suggest treatment of the present invention for a patient.

### Pharmaceutical Composition

A pharmaceutical composition of the invention comprises at least one type of viral vector of the invention. Preferably, the present invention provides a pharmaceutical composition containing (a) an oncolytic virus as such or in combination with (b) adoptive cell composition or (c) a checkpoint inhibitor. The present invention also provides said pharmaceutical combination for use in the treatment of cancer. Furthermore, the composition may comprise at least two, three or four different vectors. In addition to the vector and adoptive cell composition or checkpoint inhibitor, a pharmaceutical composition may also comprise other therapeutically effective agents, any other agents such as pharmaceutically acceptable carriers, buffers, excipients, adjuvants, additives, preservatives, antiseptics, filling, stabilising and/or thickening agents, and/or any components normally found in corresponding products. Selection of suitable ingredients and appropriate manufacturing methods for formulating the compositions belongs to general knowledge of a man skilled in the art.

The pharmaceutical composition may be in any form, such as solid, semisolid or liquid form, suitable for administration. A formulation can be selected from a group consisting of, but not limited to, solutions, emulsions, suspensions, tablets, pellets and capsules. The compositions of the current invention are not limited to a certain formulation, instead the composition can be formulated into any known pharmaceutically acceptable formulation. The pharmaceutical compositions may be produced by any conventional processes known in the art.

A pharmaceutical kit of the present invention comprises an oncolytic adenoviral vector encoding a variant IL-2 as a transgene and one or more immune checkpoint inhibitors. The oncolytic adenoviral vector encoding a variant IL-2 as a transgene is formulated in a first formulation and said one or more immune checkpoint inhibitors are formulated in a second formulation. Alternatively, the pharmaceutical kit of the present invention comprises an oncolytic adenoviral vector encoding a variant IL-2 as a transgene in the first formulation and an adoptive cell composition in the second formulation. In another embodiment of the invention the first and the second formulations are for simultaneous or sequential, in any order, administration to a subject. In another embodiment, said kit is for use in the treatment of cancer or tumor.

### Administration

The vector or pharmaceutical composition of the invention may be administered to any mammal subject. In a specific embodiment of the invention, the subject is a human. A mammal may be selected from a group consisting of pets, domestic animals and production animals.

Any conventional method may be used for administration of the vector or composition to a subject. The route of administration depends on the formulation or form of the composition, the disease, location of tumors, the patient, comorbidities and other factors. Accordingly, the dose amount and dosing frequency of each therapeutic agent in the combination depends in part on the particular therapeutic agent, the severity of the cancer being treated, and patient characteristics. Preferably, a dosage regimen maximizes the amount of each therapeutic agent delivered to the patient consistent with an acceptable level of side effects.

The effective dose of vectors depends on at least the subject in need of the treatment, tumor type and location of the tumor and stage of the tumor. The dose may vary for example from about 1×10⁸ viral particles (VP) to about 1×10¹⁴ VP, specifically from about 5×10⁹ VP to about 1×10¹³ VP and more specifically from about 3×10⁹ VP to about 2×10¹² VP. In one embodiment oncolytic adenoviral vectors coding for a variant IL-2 are administered in an amount of 1×10¹⁰-1×10¹⁴ virus particles. In another embodiment of the invention the dose is in the range of about 5×10¹⁰-5×10¹¹ VP.

In one embodiment of the invention, the administration of oncolytic virus is conducted through an intratumoral, intra-arterial, intravenous, intrapleural, intravesicular, intracavitary, intranodal or peritoneal injection, or an oral administration. Any combination of administrations is also possible. The approach can give systemic efficacy despite local injection.

In one embodiment of the invention, the separate administration(s) of (a) an oncolytic adenoviral vector encoding a variant IL-2 as a transgene and (b) one or more immune checkpoint inhibitors to a subject is (are) conducted simultaneously or consecutively, in any order. This means that (a) and (b) may be provided in a single unit dosage form for being taken together or as separate entities (e.g. in separate containers) to be administered simultaneously or with a certain time difference. This time difference may be between 1 hour and 2 weeks, preferably between 12 hours and 3 days, more preferably up to 24 or 48 hours. In a preferred embodiment, the first administration of the adenoviral vector is conducted before the first administration of the checkpoint inhibitor. In addition, it is possible to administer the virus via another administration way than the checkpoint inhibitor. In this regard, it may be advantageous to administer either the virus or checkpoint inhibitor intratumorally and the other systemically or orally. In a particular preferred embodiment, the virus is administered intratumorally and the checkpoint inhibitor intravenously. Preferably, the virus and the checkpoint inhibitor are administered as separate compounds. Concomitant treatment with the two agents is also possible.

In a preferred embodiment, the checkpoint inhibitor is administered in an amount from about 2 mg/kg to 50 mg/kg, more preferably about 2 mg/kg to 25 mg/kg.

As used herein “separate administration” or “separate” refers to a situation, wherein (a) an oncolytic adenoviral vector encoding a variant IL-2 as a transgene and (b) one or more immune checkpoint inhibitors are two different products or compositions distinct from each other.

Any other treatment or combination of treatments may be used in addition to the therapies of the present invention. In a specific embodiment the method or use of the invention further comprises administration of concurrent or sequential radiotherapy, chemotherapy, antiangiogenic agents or targeted therapies, such as alkylating agents, nucleoside analogs, cytoskeleton modifiers, cytostatic agents, monoclonal antibodies, kinase inhibitors or other anti-cancer drugs or interventions (including surgery) to a subject.

The terms “treat” or “increase”, as well as words stemming therefrom, as used herein, do not necessarily imply 100% or complete treatment or increase. Rather, there are varying degrees of which one of ordinary skill in the art recognizes as having a potential benefit or therapeutic effect.

It will be obvious to a person skilled in the art that, as the technology advances, the inventive concept can be implemented in various ways. The invention and its embodiments are not limited to the examples described above but may vary within the scope of the claims.

### Experimental Section

### Materials and Methods

### Cell Lines

Human lung adenocarcinoma A549, human melanoma SK-MEL-28 and hamster leiomyosarcoma DDT1-MF2 cell lines were maintained in DMEM and hamster pancreatic cancer HapT1 was maintained in RPMI. Both DMEM or RPMI were supplemented with 10% fetal bovine serum (FBS), 100 U/mL penicillin, 100 mg/mL streptomycin, and 2 mM L-glutamine (all from Sigma-Aldrich). Both cell lines were cultured at +37° C. and 5% CO₂.

### Recombinant Human Cytokine

Recombinant human (rh) IL-2 (Peprotech) and rh vIL-2 (Adipogen) cytokines were used as positive controls in the ex vivo experiments in concentrations of 0.1-100 U/mL.

Virus and vIL-2 Transgene Construction

All the viruses used in this study have the backbone of Ad5/3-E2F-d24. The construction of this and Ad5/3-E2F-d24-IL-2 has been explained previously (Havunen et al., 2017).

The vIL-2 transgene was constructed by making five point mutations in IL-2 sequence at positions 80 L->F, 81 R->D, 85 L->V, 86 I->V and 92 I->F. Ad5/3-E2F-d24-vIL-2 virus was generated with bacterial artificial chromosome (BAC)-recombineering strategy, which used galk selection (Warming et al., 2005; Muck-Hausl et al., 2015). The transgene vIL-2 was inserted in E3 region by homologous recombination. PCR-amplified vIL-2 was electroporated into SW102 bacteria containing BAC-Ad5/3-E2F-Δ24-GalK/amp and the positive clones with vIL-2 transgene were identified with deoxyglucose selection. The sequence was verified by restriction enzyme analysis. The virus genome was released from BACs with Pad restriction enzyme (Thermo Scientific) and transfected into A549 cells with Lipofectamine 2000 reagent (Invitrogen). The vIL-2-armed Ad5/3 virus was then purified twice with cesium chloride gradient centrifugation. Optical density and tissue culture infectious dose (TCID50) assay was used to determine viral particle (VP) concentration and infectious units, respectively.

### Cytokine Expression by Virus Ex Vivo

A549 cells were infected with either Ad5/3-E2F-d24-IL-2, Ad5/3-E2F-d24-vIL-2, or left uninfected for 48 hr. Supernatant was collected and filtered (Amicon ultra 100K), and then analyzed with IL-2 human ELISA kit (Abcam) according to the manufacturer's instructions to determine the amount of virally-produced cytokines.

### Lytic Potency Assay

10,000 A549 cells/well were plated in 100ul of 2% DMEM assay media into 96-well plate. Cells were infected with Ad5/3-E2F-d24, Ad5/3-E2F-d24-IL-2, or Ad5/3-E2F-d24-vIL-2 at 0-1000 VP/cell in triplicates. After 3 days, cell viability was determined with MTS cytotoxicity assay according to manufacturer's instructions (cell titer 96 Aqueous One Solution Cell Proliferation Assay, Promega, Madison, WI).

### Cell Proliferation Assay

Peripheral blood mononuclear cells (PBMCs) were obtained from healthy donors and isolated through density gradient centrifugation using Lymphoprep (StemCell technologies). The PBMCs were incubated with rh vIL-2 and rh IL-2 at different concentrations (0.1 U, 1 U, 10 U, and 100 U) for three days and analyzed for CD4+ T cells, CD8+ T cells, and NK cells through flow cytometry. To measure the relative cell expansion, we compared the percentage of positive cells on day 3 to the corresponding numbers on day 0.

### T-Cell Isolation, and Stimulation

T cells were enriched from freshly isolated PBMCs through CD3+ T cell isolation kit (Miltenyi Biotec). Sorted T lymphocytes were activated with CD3/CD28 beads (Invitrogen) in a 1:5 bead/T-cell ratio and then cultured for 4 days either with: (1) rh IL-2 at 100 U/mL; (2) rh vIL-2 at 100 U/mL, or (3) without any cytokine, but with complete media as a control. These three conditions were studied in three groups: in group one, activated T cells only; in group two, tumor cells in addition to activated T cells; and in group three, activated T cells and tumor cells with unarmed virus Ad5/3-E2F-d24. Cytokines and half of the assay medium were replaced on day 2. Cells were analyzed on days 0, 2, and 4 by flow cytometry with Sony SH800Z (Sony, Tokyo, Japan).

Immune Subset Analysis after Virus Infection Ex Vivo

Tumor cells were infected with either unarmed Ad5/3-E2F-d24, Ad5/3-E2F-d24-IL-2, or Ad5/3-E2F-d24-vIL-2 viruses at 100 VP/cell or left uninfected. PBMCs isolated from healthy donor were added on top of infected cancer cells 24 hours post-infection. PBMCs alone were used as mock control. Cells were stained immunofluorescently with anti-CD3, anti-CD8, anti-CD4, anti-CD25, anti-CD69, anti-CD127, and anti-CD56 and analyzed on days 0, 3, and 6 through BD Accuri C6 flow cytometer. Next, the effects of specific immune cell populations, namely T cells and NK cells, were studied more in detail in a similar set up.

### Animal Experiment

To study treatment-induced changes in tumors, 2*10⁶ HapT1 cells per animal were implanted on the lower back subcutaneously in 5 week-old immunocompetent Syrian hamsters. Animals were randomized into groups of four (n=13), when the average tumor diameter reached 0.5 cm. Viruses Ad5/3-E2F-d24, Ad5/3-E2F-d24-IL-2, and Ad5/3-E2F-d24-vIL-2 were administered intratumorally at 1*10⁹ VPs and mock received PBS only. Virus were injected on days 1, 4, 8, and 13.

Five animals were euthanized from each group on day 16 and tumors and selected organs were collected to evaluate histopathological characteristics and immune cell subsets present. The rest of the animals were monitored for survival. These animals received 6 additional rounds of virus treatment after every 5 days, starting at day 18. Tumors were measured with digital caliper in all even days till day 30. End point criteria included 20.0 mm tumor size limit and skin ulcerations.

Cured animals were re-challenged on their upper back with either the same HapT1 tumor (2*10⁶ cells/tumor) or with a different tumor DDT-MF2 (1.5*10⁵ cells/tumor) after the observation period of 160 days. Naïve animals (n=3) that had not been exposed to any cancer cell or treatment before were included as mock group. Tumor growth was followed for 21 days until DDT1-MF2 tumors reached the maximum tolerated diameter. Of note, two out of three Ad5/3-E2F-d24 therapeutic animals were not re-challenged because of the presence of visible tumors, i.e. the tumors had not been cured with unarmed virus.

### Histopathology

For pathological evaluation, hamster organs such as liver, spleen, lung, kidney, heart, and tumor samples were collected on day 16 from five hamsters of each group. Collected samples were first fixed in 10% formalin, after 48 hr transferred to 70% ethanol and embedded in paraffin. For microscopic evaluation, tissue sections with 5 μm thickness were stained with hematoxylin and eosin. A pathologist evaluated the histological changes in stained tissue samples.

### Statistical Analysis

Evaluation of tumor growth was performed with Linear mixed model with the log-transformed tumor volumes with SPSS version 25 Statistics (IBM). Two-way ANOVA and Log-rank (Mantel-Cox) tests were used to analyse the group variation in the re-challenged and survival curve, respectively. GraphPad Prism (version 8.0.0.) was used to present individual and grouped tumor growth data and to plot survival curve. P value was considered significant when p<0.05.

### Example 1. Effector Cells Proliferate More in the Presence of vIL-2 than with Conventional IL-2 Ex Vivo

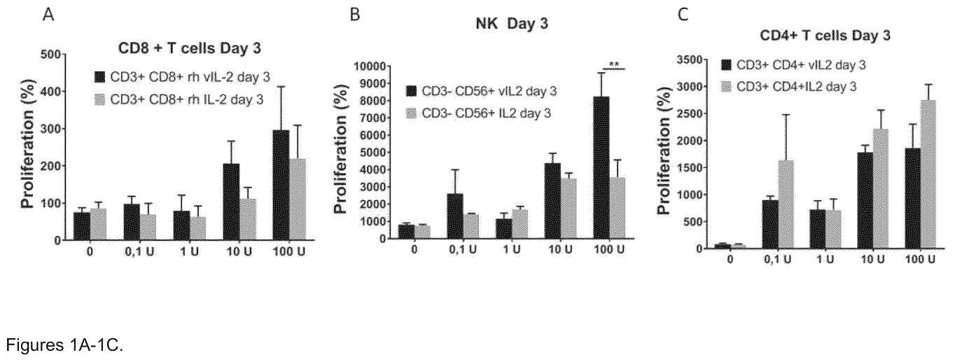

We compared rh vIL-2 and rh IL-2 with regard to their ability to stimulate immune cells, such as CD8+ T cells, NK cells, and CD4+ T cells. We cultured PBMCs either with or without recombinant human (rh) vIL-2 or rh IL-2 at different concentrations (0.1-100 U/ml). After 3 days, the rh vIL-2 was more potent in inducing the proliferation of CD8+ effector T cells and NK cells than IL-2, whereas the levels of CD4+ T cells (including Tregs) remained lower with the variant (FIG. 1). These results indicated that the vIL-2 has preferred effects on T cells and NK cells over the conventional IL-2. It should be noted that as activated T-cells produce IL-2, there will be IL-2 present in cultures even in the vIL-2 groups. This would be expected to dilute the (lack of) effect of vIL-2 on Tregs, for example.

### Example 2. The Effects of Rh vIL-2 on Different T Cell Subsets Provides Rationale for Constructing a Virus Coding for the Cytokine

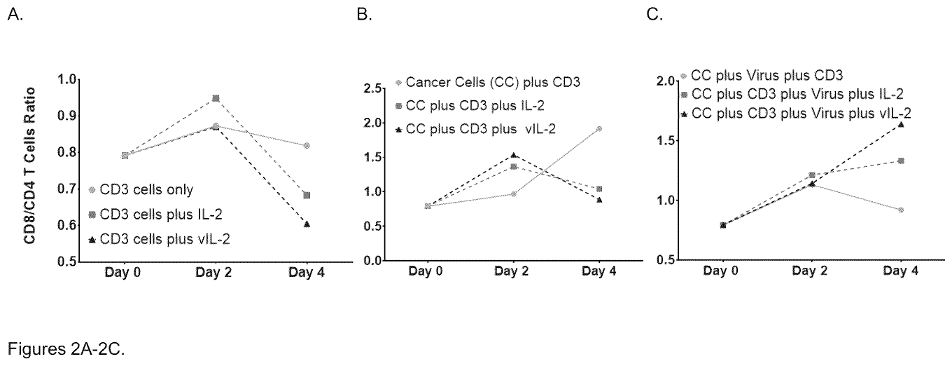

To investigate the effect of rh vIL-2 on T cells in the presence of adenovirus, we isolated T cells with CD3/CD28 beads and activated them for 4 days with either 100 U/ml of rh vIL-2 or rh IL-2 and infected/non-infected cancer cells. IL-2 and vIL-2 had similar effects on CD8/CD4 cell ratios in the presence and absence of cancer cells (FIGS. 2A and B). However, when the cancer cells were infected with an oncolytic virus, vIL-2 induced a trend towards CD8+CD27-CD62L-CD45RO+ cell dominance over CD4+ cells (FIG. 2C).

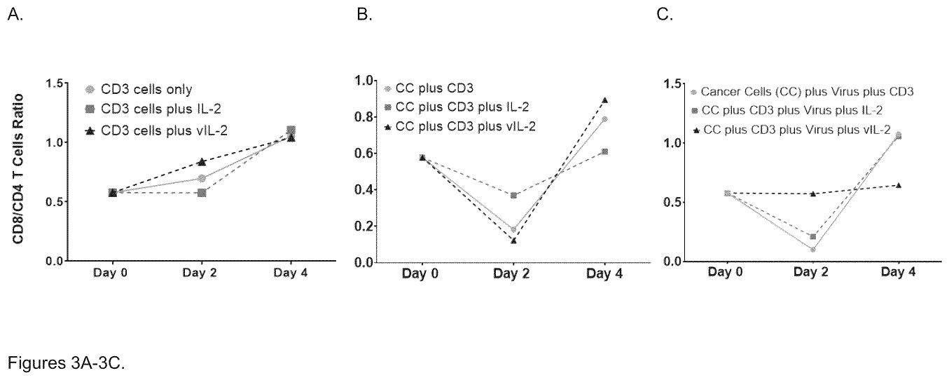

The hallmark of acquired immunity is a memory response, which is the consequence of antigen-specific lymphocytes' clonal expansion and differentiation that persists for a lifetime (Sallusto et al., 2004). We evaluated the percentage change of central memory T cells (Tcm; CD45RO+, CD62L+, CD27+) in the presence of rh IL-2 or rh vIL-2 with infected or non-infected cancer cells. Tcm mediate reactive memory responses and differentiate to effector cells upon antigen stimulation. We did not find any difference in CD8/CD4 Tcm ratios between IL-2 and vIL-2 in the absence of cancer cells (FIG. 3A). In the presence of tumor cells, we first observed a decrease in the ratio on day 2, followed by an increase on day 4. Again, the vIL-2 induced higher CD8 to CD4 ratio in the Tcm population than the conventional IL-2 (FIG. 3B).

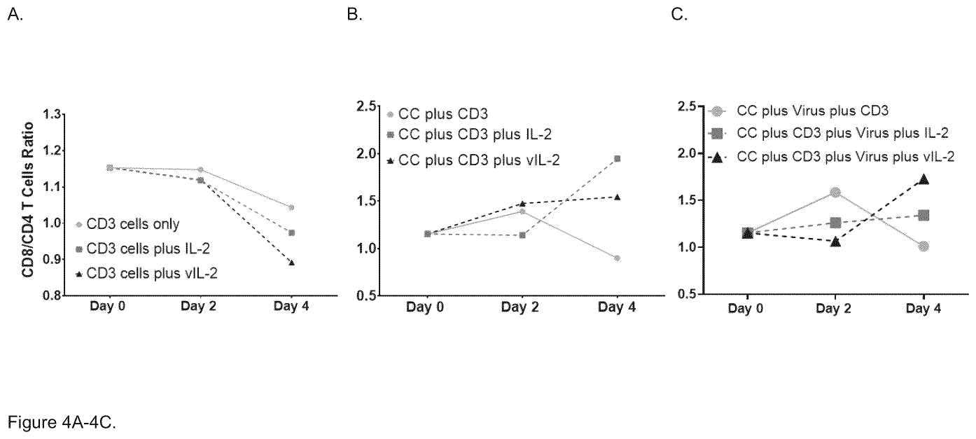

In addition to Tcm, we also evaluated effector memory T cells (Tem; CD45RO+, CD62L-, CD27+) in the same conditions as Tcm cells. Tem provide protective memory and are characterized by prompt effector function. We did not observe differences in CD8/CD4 Tem ratio between IL-2 and vIL-2, if cancer cells were not present (FIG. 4A). With cancer cells, IL-2 and vIL-2 induce high ratio of CD8/CD4 Tem by day 4 (FIG. 4B). When the cancer cells were infected, we observed a trend towards high CD8/CD4 Tem ratio in rh vIL-2 group on day 4 (FIG. 4C). To conclude, we did not see significant difference between rh IL-2 and rh vIL-2 with regard to their effect on pro-inflammatory T cell compartments. These results provided us solid grounds to construct a vIL-2 armed oncolytic adenovirus.

### Example 3. Ad5/3-E2F-d24-vIL-2 Virus Expresses vIL-2 and Kills Tumor Cells Efficiently Ex Vivo

Adenovirus 5/3 features the backbone of adenovirus serotype 5 and fiber knob of adenovirus serotype 3, to enhance tumor transduction, as its receptor is highly expressed in advanced tumors (Wang et al., 2011). To restrict virus replication to tumor cells, a mutation in constant region 2 of the E1A gene and introduction of a heterologous tumor-specific E2F promoter were performed. To enhance apoptosis enabling deletion of E1B 19K gene region was made. The variant IL-2 transgene was placed into the E3 region under the E3 promoter, to link the expression to virus replication (FIG. 5A). The transgene cassette replaces the open reading frames for gp19k and 6.7k.

To investigate the oncolytic potency of the constructed virus, cytotoxicity assay was performed using human lung cancer A549 cells. There were no major differences between viruses' cell killing ability between Ad5/3-E2F-d24-IL-2 and Ad5/3-E2F-d24-vIL-2, thus indicating that presence of vIL-2 transgene does not reduce the oncolytic potency of the virus (FIG. 5B). Additionally, cells infected with Ad5/3-E2F-d24-vIL-2 were able to secrete the cytokine (FIG. 5C).

### Example 4. Ad5/3-E2F-d24-vIL-2 Stimulates Effector Cells but not Tregs Ex Vivo

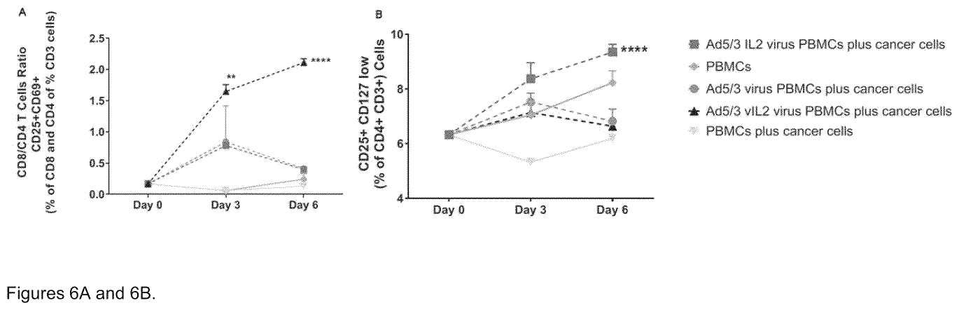

Human cancer cells A549 were infected with either Ad5/3-E2F-d24, Ad5/3-E2F-d24-IL-2, or Ad5/3-E2F-d24-vIL-2, or left uninfected. After 24h, cancer cells were incubated with PBMCs. The CD8/CD4 ratio of CD25+CD69+activated effector T cells was significantly higher in the group treated with Ad5/3-E2F-d24-vIL-2 on day 3 and day 6, than when treated with the virus expressing conventional IL-2 (FIG. 6A). Actually, we did not see any significant difference in CD8/CD4 ratio of activated effector T cells between the control viruses. Thus, vIL-2-armed Ad5/3 virus is a potent stimulator for effector cells.

To investigate effects on Tregs, we analyzed CD25+CD127^{low} expressing cells of the CD4+CD3+parent population. Ad5/3-E2F-d24-vIL-2 did not induce Treg differentiation like Ad5/3-E2F-d24-IL-2 (FIG. 6B). Thus, virally produced vIL-2 seems to retain the key attractive feature of recombinant vIL-2: preferential stimulation of effector cells over Tregs.

### Example 5. Variant IL-2 Armed Oncolytic Adenovirus Significantly Enhances Antitumor Efficacy and Survival in Hamsters

Following the promising ex vivo results, variant IL-2 armed adenovirus was then studied in immunocomptent Syrian hamsters. Since human adenoviruses are able to replicate in hamsters (unlike in mice) and some human cytokines such as human IL-2 are bioactive in hamsters (Havunen et al., 2017; Gowen et al., 2008), it is the optimal model for studying armed oncolytic adenoviruses (Havunen et al., 2017).

Animals treated with backbone Ad5/3-E2F-d24 or IL-2 armed virus, (Ad5/3-E2F-d24-IL-2) showed a trend for tumor control as compared to mock (difference not significant). Impressively, we got best tumor control in the group treated with Ad5/3-E2F-d24-vIL-2 and this result was statistically significant in comparison to all other groups by day 30. This underlines the utility of vIL-2 as a stimulator of anti-tumor effector T cells without the unwanted immunosuppressive effects on Treg. Thus, Ad5/3-E2F-d24-vIL-2 appears to be a potent modulator of the tumor microenvironment towards a direction compatible with complete tumor eradication.

In order to investigate the mechanism of action of the therapy, we treated hamsters with either backbone Ad5/3-E2F-d24, Ad5/3-E2F-d24-IL-2, Ad5/3-E2F-d24-vIL-2 or PBS on days 1, 4, 8, and 13. On day 16, hamsters were euthanized, tumors were collected to deeply analyze tumor microenvironment through flow cytometry and Nanostring assessments. To study treatment-related changes, tumors and selected organs were collected for histopathological evaluation. Pathological results revealed no difference between mock and oncolytic adenovirus treated groups thus, our virus didn't cause any systemic toxic effects.

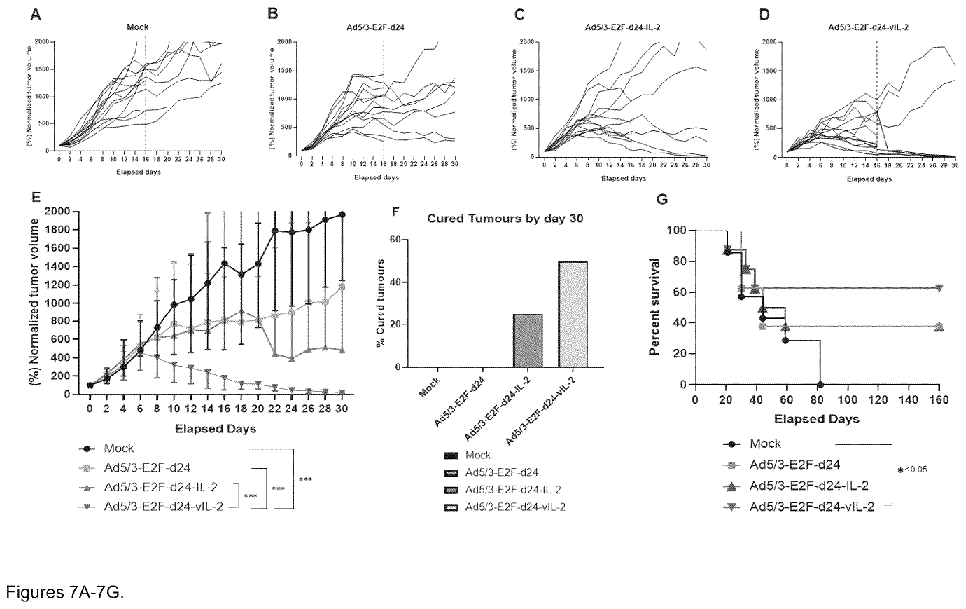

Survival data shows that the group treated with backbone virus was able to cure one hamster. Additionally, two hamsters with stable tumors survived to the end of the experiment. Ad5/3-E2F-d24-IL-2 cured three hamsters. Ad5/3-E2F-d24-vIL-2 was able to cure 60% of treated hamsters and this difference was significant over mock (FIG. 7).

### Example 6. Treatment with Cytokine Armed Adenovirus Induces Tumor-Specific Immunological Memory

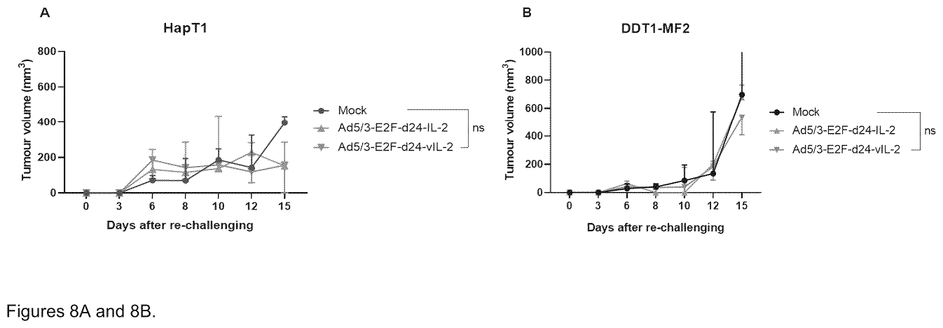

In order to study if oncolytic virus treatment had induced tumor-specific immunological memory, all cured hamsters were re-challenged with the same HapT1 cancer cells and with different DDT1-MF2 cancer cells. Both cancer cell types were implanted on the upper back of cured hamsters. In this re-challenge experiment, the number of animals per group differed because different viruses had cured a different number of hamsters. There was no hindrance of HapT1 (FIG. 8A) or DDT1-MF2 (FIG. 8B) tumor rechallenge in naïve or unarmed virus treated animals. In contrast, prior cure of HapT1 with Ad5/3-E2F-d24-IL-2 or Ad5/3-E2F-d24-vIL-2 appeared to offer protection against HapT1 rechallenge. Of note, 40% of animals treated with Ad5/3-E2F-d24-vIL-2 remained HapT1 tumor free after rechallenged. However, DDT-MF2 tumors grew normally in these animals underlining the epitope-specific immunological memory induced by the armed viruses. These findings are in line with previous data indicating the importance of cytokines such as IL-2 in establishing systemic and tumor-specific antitumor immunity (Havunen et al., 2017).

We have shown that variant IL-2 armed adenovirus Ad5/3-E2F-d24-vIL-2 appears safe and effective in immunocompetent Syrian hamsters. Ad5/3-E2F-d24-vIL-2 exhibited potent antitumor efficacy and expression of variant IL-2 within the tumor microenvironment. Studies on human lymphocytes indicated induction of antitumor immunological effects. Specifically, vIL-2 seemed to preferentially activate effector T-cells over suppressive Tregs.

### Example 7. Variant IL-2 Coding Oncolytic Adenovirus Induces Substantial Tumor Reduction with Moderate Infiltration of Immune Cells and the Highest Intratumoral Expression of IL-2

### Cell Lines

Hamster pancreatic cancer HapT1 was maintained in RPMI supplemented with 10% fetal bovine serum (FBS), 100 U/mL penicillin, 100 mg/mL streptomycin, and 2 mM L-glutamine (all from Sigma-Aldrich). Both cell lines were cultured at +37C° and 5% CO₂.
Virus and vIL-2 Transgene Construction
All the viruses used in this study have the backbone of Ad5/3-E2F-d24. The construction of the latter and Ad5/3-E2F-d24-IL-2 has been explained previously in Havunen et al., 2017. The vIL-2 transgene was constructed by making five point mutations in IL-2 sequence at positions 80 L->F, 81 R->D, 85 L->V, 86 I->V and 92 I->F. Ad5/3-E2F-d24-vIL-2 virus was generated with bacterial artificial chromosome (BAC)-recombineering strategy, which used galk selection (Warming et al., 2005; Muck-Hausl et al., 2015). The transgene vIL-2 was inserted in E3 region by homologous recombination. PCR-amplified vIL-2 was electroporated into SW102 bacteria containing BAC-Ad5/3-E2F-Δ24 -GalK/amp and the positive clones with vIL-2 transgene were identified with deoxyglucose selection. The sequence was verified by restriction enzyme analysis. The virus genome was released from BACs with PacI restriction enzyme (Thermo Scientific) and transfected into A549 cells with Lipofectamine 2000 reagent (Invitrogen). The vIL-2-armed Ad5/3 virus was then purified twice with cesium chloride gradient centrifugation. Optical density and tissue culture infectious dose (TCID50) assay was used to determine viral particle (VP) concentration and infectious units, respectively.

### Animal Experiment

To study treatment-induced changes in tumors, 2×10⁶ HapT1 cells per animal were implanted on the lower back subcutaneously in 5 week-old immunocompetent Syrian hamsters. Animals were randomized into groups of four (n=13), when the average tumor diameter reached 0.5 cm. Viruses Ad5/3-E2F-d24, Ad5/3-E2F-d24-IL-2, and Ad5/3-E2F-d24-vIL-2 were administered intratumorally at 1×10⁹ VPs and mock received PBS only. Viruses were injected on days 1, 4, 8, and 13.

Five animals were euthanized from each group on day 16 and tumors were collected to evaluate immunological changes and mRNA expression levels.

### Flow Cytometry

Hamster tumour samples collected on day 16, were processed as single cell suspensions and further analyzed following previously established protocols (Havunen et al., 2017; Siurala et al., 2016) These samples were then stained with antibodies for CD8+(PE, 12-0080-82), CD4+(PE-Cyanine 7, 25-0041-82), and WIC II+ cells (FITC, 11-5980-82) cells. NK+ cells were labelled with the polyclonal antibody anti-Asialo-GM1 (Alexa Fluor-488, 53-6507-80), and macrophages+ cells with anti-Galectin (PE, 12-5301-82) as described before (Havunen et al., 2017). Cell fluorescence was detected using Sony SH800Z cytometer (Sony, Tokyo, Japan) upon the acquisition of the 100.000 events per sample. Cell data processing and gating were performed with FlowJo v.10.6.1 (BD®, New Jersey, USA).

### Gene-Expression Analysis

Fragments of animal tumour samples harvested on day 16 were preserved in RNAlater (R0901; Sigma-Aldrich, St. Louis, USA), and stored in −20° C. until further processing. RNA from these samples were then purified with RNAeasy Mini Kit (74104; QIAGEN, Hilden, Germany) following the manufacturer's instructions. The final RNA yield was measured with the Thermo Scientific NanoDrop™ 1000 Spectrophotometer (Thermo Fisher Scientific, Massachusetts, USA), and the RNA concentration of the samples were adjusted to 20 ng/μ1.
Reverse Transcriptase (RT)-Quantitative Polymerase Chain Reaction (qPCR)
RNA purified from Day 16 tumours, were used to synthetize cDNA using Quantitect Reverse Transcription Kit (205313, QIAGEN, Hilden, Germany) to be used for the relative quantification of the viruses transgene expression as well as for hamster IL-2 relative expression. The reverse transcription real-time PCR (RT-qPCR) was performed as previously described by Santos et al., 2017. Wild-type IL-2 virus transgene was detected with the primers and probe designed for human IL-2. For IL-2 variant virus transgene, primers and probe designed for humanIL-2v were used. Normalization of hamster IL-2 and viruses transgenes were performed using hamster GAPDH gene (Siurala et al., 2015).

### Statistical Analysis

GraphPad Prism (version 8.0.0.) was used to present tumor volume data, relative and absolute mRNA expression levels. Unpaired t-test with Welch's correction was performed to assess differences between different therapeutic groups. Pearson's correlation coefficient was calculated to determine correlation between granzyme production with SAP gene or variant IL-2 transgene production. P value was considered significant when p<0.05.

### Results

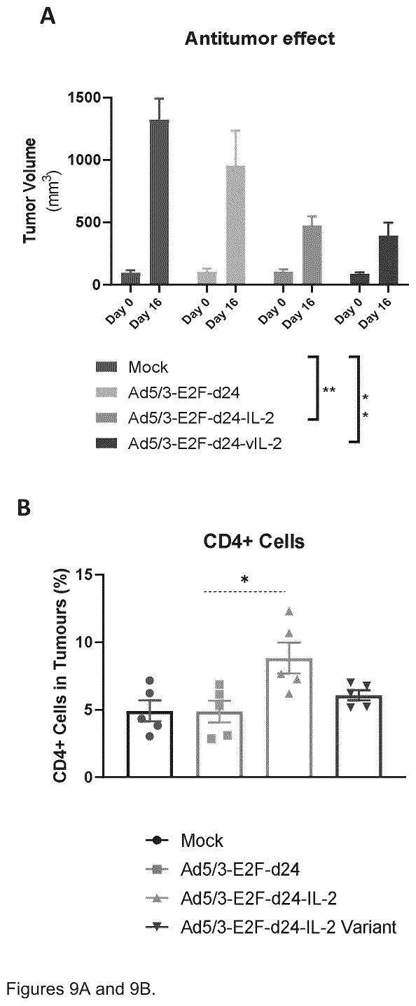

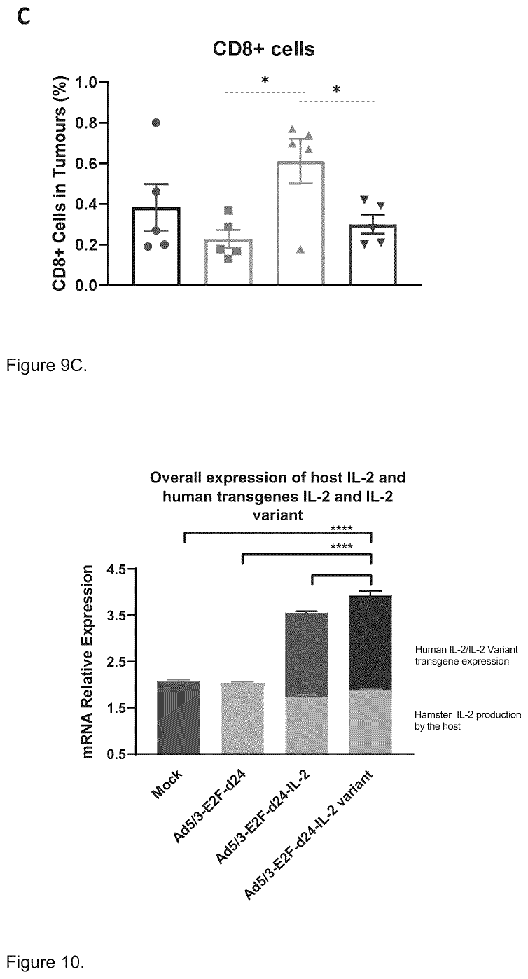

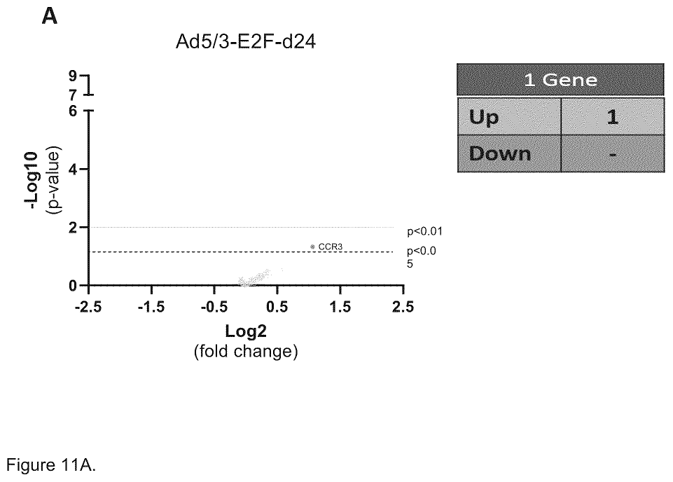

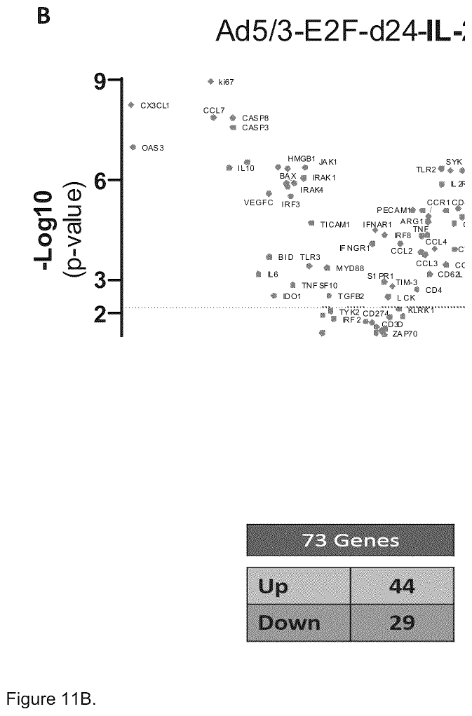

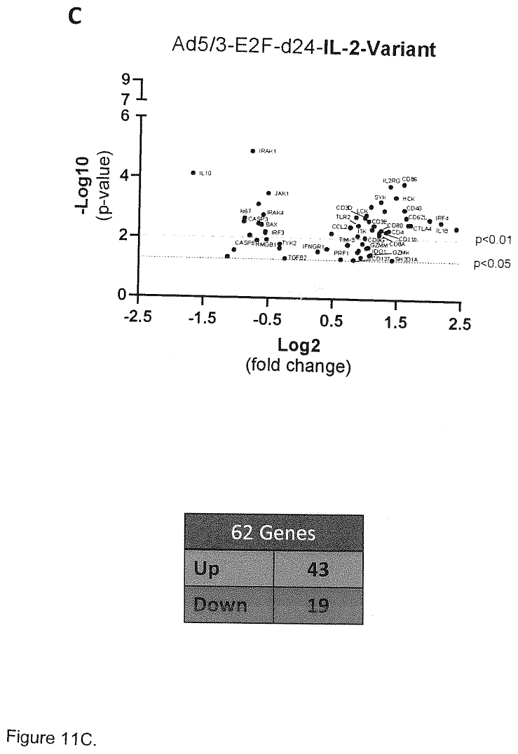

To gain further insight on the biological events induced by the oncolytic adenovirus treatments, we collected tumors 16 days after the experiment started. Comparison of tumor volumes from day 0 and 16 showed that treatment with Ad5/3-E2F-D24 had a minimum effect in controlling the growth of hamster tumors (FIG. 9A). On the other hand, human IL-2 and variant IL-2-coding oncolytic adenovirus treatments substantially delayed tumor growth of hamster pancreatic tumors. Such effect was more prominent in hamsters treated with variant IL-2-coding oncolytic adenoviruses considering that, by day 16, this group demonstrated a trend towards the lowest tumor burden (FIG. 9A).
Further analysis of the immunological compartment revealed that the frequency of CD4+ and CD8+ cells in tumors was the highest in the wild-type human IL-2 oncolytic adenovirus treated hamsters (FIG. 9B-C). In contrast, tumors from hamsters undergoing variant IL-2-virus therapy had surprisingly moderate levels of infiltration of these cells (FIG. 9B-C). Considering that no significant difference in the tumor volumes on day 16 was observed between the cytokine-armed viruses, this suggests that the virus-expressing variant IL-2 induces qualitatively superior antitumor response relative to the wild-type counterpart.
In addition, key limitations of wild-type human IL-2 are its pharmacokinetics (short half-life) and, consequently, its minimal accumulation at the target lesions (Arenas-Ramirez et al., 2015). The results in FIG. 10 demonstrate that the above-mentioned problems can be ablated with the Ad5/3-E2F-D24-vIL-2 oncolytic adenovirus. For example, a significant difference can be observed in the production of IL-2 between tumors from hamsters treated with wild-type IL-2 or variant IL-2 oncolytic adenoviruses (FIG. 10). In fact, Ad5/3-E2F-D24-vIL-2 treated group presented the highest global cytokine expression (host IL-2 and IL-2 variant transgene) compared to all other groups. Moreover, the levels of IL-2 variant were high even after 3 days of the last virus-treatment (FIG. 10). It is important to note that these results are unforeseen for proteins of similar nature, which is especially remarkable, considering that the effort to develop recombinant variant IL-2 proteins, focuses on improving safety and intratumorally relevant high-levels of variant IL-2 proteins (Arenas-Ramirez et al., 2015).

### Example 8. Variant IL-2 Coding Oncolytic Adenovirus Therapy Causes Immune Reconfiguration for Increased Effector T-Cell Function and Low Immunosuppression

### Cell Lines

Hamster pancreatic cancer HapT1 was maintained in RPMI supplemented with 10% fetal bovine serum (FBS), 100 U/mL penicillin, 100 mg/mL streptomycin, and 2 mM L-glutamine (all from Sigma-Aldrich). Both cell lines were cultured at +37C° and 5% CO₂.
Virus and vIL-2 Transgene Construction
All the viruses used in this study have the backbone of Ad5/3-E2F-d24. The construction of the latter and Ad5/3-E2F-d24-IL-2 has been explained previously in Havunen et al., 2017. The vIL-2 transgene was constructed by making five point mutations in IL-2 sequence at positions 80 L->F, 81 R->D, 85 L->V, 86 I->V and 92 I->F. Ad5/3-E2F-d24-vIL-2 virus was generated with bacterial artificial chromosome (BAC)-recombineering strategy, which used galk selection (Warming et al., 2005; Muck-Hausl et al., 2015). The transgene vIL-2 was inserted in E3 region by homologous recombination. PCR-amplified vIL-2 was electroporated into SW102 bacteria containing BAC-Ad5/3-E2F-Δ24-GalK/amp and the positive clones with vIL-2 transgene were identified with deoxyglucose selection. The sequence was verified by restriction enzyme analysis. The virus genome was released from BACs with PacI restriction enzyme (Thermo Scientific) and transfected into A549 cells with Lipofectamine 2000 reagent (Invitrogen). The vIL-2-armed Ad5/3 virus was then purified twice with cesium chloride gradient centrifugation. Optical density and tissue culture infectious dose (TCID50) assay was used to determine viral particle (VP) concentration and infectious units, respectively.

### Animal Experiment

To study treatment-induced changes in tumors, 2×10⁶ HapT1 cells per animal were implanted on the lower back subcutaneously in 5 week-old immunocompetent Syrian hamsters. Animals were randomized into groups of four (n=13), when the average tumor diameter reached 0.5 cm. Viruses Ad5/3-E2F-d24, Ad5/3-E2F-d24-IL-2, and Ad5/3-E2F-d24-vIL-2 were administered intratumorally at 1×10⁹ VPs and mock received PBS only. Viruses were injected on days 1, 4, 8, and 13.
Five animals were euthanized from each group on day 16 and tumors were collected to evaluate immunological changes and mRNA expression levels.

### Gene-Expression Analysis

Fragments of animal tumour samples harvested on day 16 were preserved in RNAlater (R0901; Sigma-Aldrich, St. Louis, USA), and stored in −20° C. until further processing. RNA from these samples were then purified with RNAeasy Mini Kit (74104; QIAGEN, Hilden, Germany) following the manufacturer's instructions. The final RNA yield was measured with the Thermo Scientific NanoDrop™ 1000 Spectrophotometer (Thermo Fisher Scientific, Massachusetts, USA), and the RNA concentration of the samples were adjusted to 20 ng/μ1.
NanoString nCounter® gene expression analysis was performed on the RNA samples from all hamster tumours utilizing the nCounter® Digital Analyzer (NanoString Technologies, Seattle, USA). Gene expression was assessed with a custom-panel designed for hamster cells containing 101 genes analysed by nSolver software 4.0 (NanoString Technologies, Seattle, USA). Differential expression is displayed as the values for each genes' gene's −log 10 (p-value) and log 2 fold change in the volcano plots. Likewise, differential expression as RNA counts (Log 2) are displayed in the bars graphs. The expression level of each gene in the treatment groups was normalized to their corresponding genes in the control (mock) group.

### Statistical Analysis

GraphPad Prism (version 8.0.0.) was used to present absolute mRNA expression levels. Unpaired t-test with Welch's correction was performed to assess differences between different therapeutic groups. Pearson's correlation coefficient was calculated to determine correlation between granzyme production with SAP gene or variant IL-2 transgene production. P value was considered significant when p<0.05.

### Results

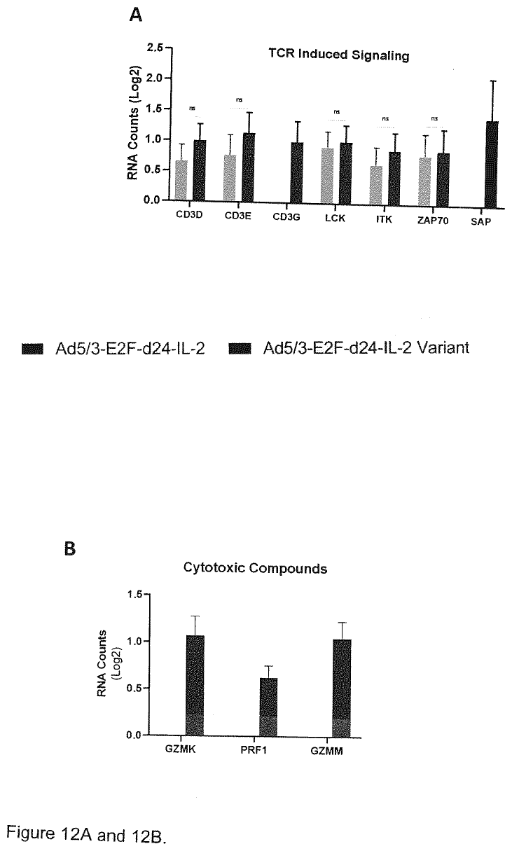

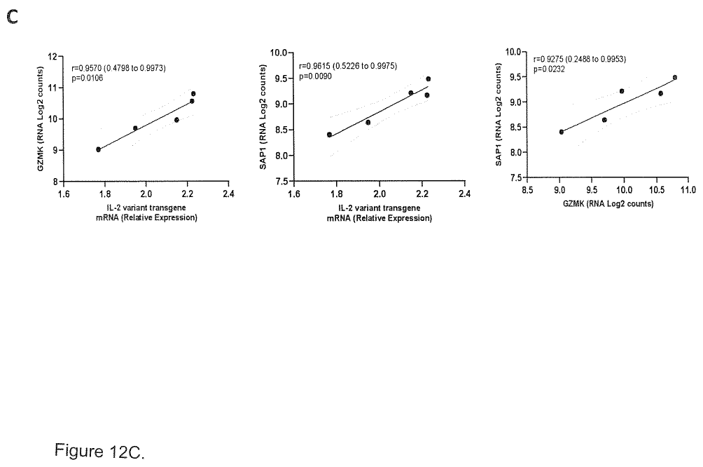

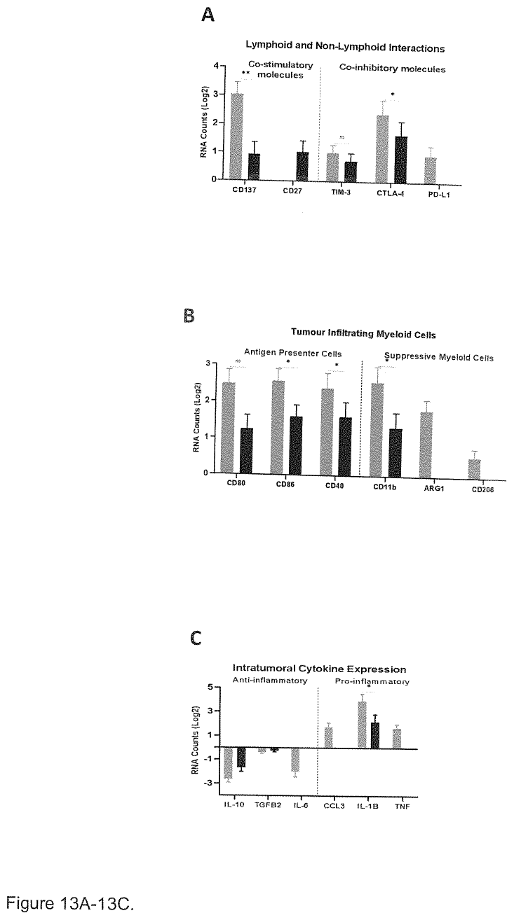

Additional characterization was performed by evaluating the transcriptome of tumors undergoing virus therapy. Compared to Ad5/3-E2F-D24 virus treatments, tumors treated with oncolytic viruses encoding wild-type IL-2 or variant IL-2 demonstrated a seemingly similar number of upregulated genes. When evaluating the downregulation profile in both groups, wild-type IL-2 had approximately 34.5% more genes downregulated than variant IL-2 group (FIG. 11). It is important to note that the wild-type IL-2 group displayed higher values for almost all genes, being up- or downregulated, compared to IL-2 variant, which is compatible with IL-2 potent biological effects previously related in the literature (FIG. 11) (Jiang et al., 2016). Here, however, elevated values did not necessarily translate into better overall response, since Ad5/3-E2F-D24-vIL-2 virus was the most successful group in promoting tumor shrinkage and overall survival.
Detailed view of the differential expression analysis, however, demonstrated that Ad5/3-E2F-D24-vIL-2 virus preferentially stimulates the expression of known genes associated to the T-cell receptor complex and downstream signaling genes, over immunosuppressive associated genes (FIGS. 12 and 13). Importantly, treatment with variant IL-2-coding oncolytic adenoviruses induces the highest expression of TCR-complex genes (CD3E, CD3D) (FIG. 12A), which are responsible for harboring the TCR upon MHC binding and initiating TCR induced signaling in humans (Ngoenkam et al., 2018). Additionally, variant IL-2-coding oncolytic adenovirus treatment upregulated known key downstream signaling genes (LCK, ITK, ZAP70) compared with the virus coding for wild-type IL-2 (FIG. 12A). Surprisingly, treatment with IL-2 variant-coding virus stimulated the upregulation of some critical genes for TCR expression, anchoring, and signaling (CD3G, SAP), while their levels for wild-type IL-2 virus group remained unaltered (FIG. 12A). Even though not statistically significant, these data suggest an unexpected increased activity of the TCR function of T-cells upon treatment with variant IL-2 coding oncolytic adenoviruses, which to our knowledge, was not documented in current art. In fact, only the variant IL-2 virus was capable of inducing high expression of granzymes and perforin (GZMK, GZMM, PRF1) (FIG. 12B). When secreted by effector cytotoxic cells (T-cells and Natural killer cells), granzymes and perforin induce apoptosis of tumor cells (Voskoboinik et al., 2015). In fact, the degree of correlation between variant IL-2 transgene mRNA relative expression and both GZMK or SAP1 genes mRNA counts is high and positive, while an additional positive correlation can be seen between GZMK and SAP1 genes (FIG. 12C). Particularly, expression of Granzyme K and Granzime M is not known from the current art to be induced by neither oncolytic adenoviruses nor variant IL-2 proteins.
The highest expression of TIM-3 and CTLA-4 genes was seen in tumors treated with wild-type IL-2-coding virus (FIG. 13A). These genes are generally known to inhibit T-cell function in humans (Ngoenkam et al., 2018), therefore they can be potentially associated with an increased immunosuppressive environment. Another gene, PD-L1, remained unaltered upon treatment with variant IL-2 virus (FIG. 13A). This may have contributed to the possible low immunosuppression in tumors belonging to the variant IL-2 virus-treated animals, considering the role of PD-L1 in immunosuppression in mice and humans. Even though the highest expression levels of CD137 (activation marker of human tumor-reactive TILs) was seen in tumors from animals treated with wild type-IL-2-coding virus (FIG. 13A), it may have been insufficient to counter the immunosuppression in these tumors. In contrast, expression of CD27 (a co-stimulatory marker) was increased in the variant-IL-2-virus groups whilst unaltered in the wild-type counterpart (FIG. 13A). Longer telomeres have been previously reported in TILs expressing CD27, thus hinting that variant IL-2 potentially increases the presence of TILs which are less terminally differentiated. On the other hand, CD27 is known to be highly expressed in memory T-cells, which have been deemed to produce a multifunctional response. This suggests that variant IL-2 oncolytic virus enables superior quality response in tumors.
On the other hand, virus-derived secretion of wild-type IL-2 caused the highest expression of known antigen-presenting cells in humans and mice (CD80, CD86, CD40) (FIG. 13B). Yet, surprisingly, wild-type IL-2-coding virus treatment rendered inferior antitumor efficacy in hamsters compared to its variant counterpart. The presence and activity (CD11b, CD206, Arg1) of immune suppressive cells, such as myeloid cells, was also reduced or unaltered in the variant IL-2 coding virus compared with the wild-type IL-2 virus (FIG. 13B). Contrary to common art, such effect is unexpected considering that certain mutations in the variant IL-2 protein aim at mainly preventing unwanted targeting of regulatory T-cells instead of other immunosuppressive cells.
Further analysis revealed an overall decreased gene expression of IL-6, TGFb, IL-10 (FIG. 13C) in tumors from hamsters treated with either wild-type IL-2 or variant IL-2-virus therapy (FIG. 13C). Interestingly, while the former had caused an increased expression of CCL3, TNF, IL-1b (FIG. 13C) (genes that encode proinflammatory cytokines in humans and mice), wild-type IL-2-virus treated tumors remained larger than the variant IL-2 virus-treated tumors. A possible explanation for this is the known ambiguous role of TNF in antitumor immunity, which in chronic levels, can contribute for the tumor progression in humans.
In summary, variant IL-2-virus promotes the ability of T-cells to harbor TCRs, signaling, lower T-cell inhibition and immunosuppression from the myeloid cells compartment. These effects may have contributed to improved cytotoxic function of effector cells, which render variant IL-2-coding oncolytic adenovirus therapy capable of providing the best survival and antitumor efficacy, compared to other therapeutic groups.

### REFERENCES

Arenas-Ramirez, N., Woytschak, J. & Boyman, O. Interleukin-2: Biology, Design and Application. Trends Immunol. 36, 763-777 (2015).
Gowen B B, Judge J W, Wong M H, Jung K H, Aylsworth C F, Melby P C, Rosenberg B, Money J D: Immunoprophylaxis of Punta Toro virus (Phlebovirus, Bunyaviridae) infection in hamsters with recombinant Eimeria profilin-like antigen. Int Immunopharmacol 2008, 8:1089-1094.
Havunen R, Siurala M, Sorsa S, Gronberg-Vaha-Koskela S, Behr M, Tahtinen S, Santos J M, Karell P, Rusanen J, Nettelbeck D M, et al.: Oncolytic Adenoviruses Armed with Tumor Necrosis Factor Alpha and Interleukin-2 Enable Successful Adoptive Cell Therapy. Mol Ther Oncolytics 2017, 4:77-86.
Itzhaki O, Levy D, Zikich D, Treves A J, Markel G, Schachter J, Besser M J: Adoptive T-cell transfer in melanoma. Immunotherapy 2013, 5:79-90.
Jiang, T., Zhou, C. & Ren, S. Role of IL-2 in cancer immunotherapy. Oncoimmunology 5, e1163462-e1163462 (2016).
Klein C, Waldhauer I, Nicolini V G, Freimoser-Grundschober A, Nayak T, Vugts D J, Dunn C, Bolijn M, Benz J, Stihle M, Lang M, Roemmele M, Hofer T, van Puijenbroek E, Wittig D, Moser S, Ast O, Brünker P, Gorr I H, Neumann S, Mudry MCV, Hinton H, Crameric F, Saro J, Evers S, Gerdes C, Bacac M, van Dongen G, Moessner E & Umafia P. (2017) Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines, OncoImmunology, 6:3, DOI: 10.1080/2162402X.2016.1277306
Levin, Aron M., Darren L. Bates, Aaron M Ring, Carsten Krieg, Jack T. Lin, Leon Su, Ignacio Moraga, Miro E. Raeber, Gregory R. Bowman, Paul Novick, Vijay S. Pande, C. Garrison Fathman, Onur Boyman and K. Christopher Garcia. Exploiting a natural conformational switch to engineer an interleukin-2 ‘superkine’. Nature vol. 484, pages 529-533 (2012).
Muck-Hausl M, Solanki M, Zhang W, Ruzsics Z, Ehrhardt A: Ad 2.0: a novel recombineering platform for high-throughput generation of tailored adenoviruses. Nucleic Acids Res 2015, 43:e50.
Ngoenkam, J., Schamel, W. W. & Pongcharoen, S. Selected signalling proteins recruited to the T-cell receptor-CD3 complex. Immunology 153, 42-50 (2018).
Sallusto F, Geginat J, Lanzavecchia A: Central memory and effector memory T cell subsets: function, generation, and maintenance. Annu Rev Immunol 2004, 22:745-763.
Santos, J. M. et al. Adenoviral production of interleukin-2 at the tumor site removes the need for systemic postconditioning in adoptive cell therapy. Int. J. Cancer 141, (2017).
Schwartz R N, Stover L, Dutcher J P: Managing toxicities of high-dose interleukin-2. Oncology (Williston Park) 2002, 16:11-20.
Siurala, M. et al. Oncolytic adenovirus and doxorubicin-based chemotherapy results in synergistic antitumor activity against soft-tissue sarcoma. Int. J. Cancer 136, 945-954 (2015).
Siurala, M. et al. Syngeneic syrian hamster tumors feature tumor-infiltrating lymphocytes allowing adoptive cell therapy enhanced by oncolytic adenovirus in a replication permissive setting. Oncoimmunology 5, e1136046—e1136046 (2016).
Sockolosky J T, Trotta E, Parisi G, Picton L, Su L L, Le A C, Chhabra A, Silveria S L, George B M, King I C, Mr T, Jude K, Sibener L V, Baker D, Shizuru J A, Ribas A, Bluestone J A, Garcia K C. (2018) Selective targeting of engineered T cells using orthogonal IL-2 cytokine-receptor complexes, Science, 359(6379): 1037, DOI: 10.1126/science.aar3246.
Tähtinen S, Blattner C, Vaha-Koskela M, Saha D, Siurala M, Parviainen S, Utikal J, Kanerva A, Umansky V, Hemminki A: T-Cell Therapy Enabling Adenoviruses Coding for IL2 and TNFalpha Induce Systemic Immunomodulation in Mice With Spontaneous Melanoma. J Immunother 2016, 39:343-354.
Voskoboinik, I., Whisstock, J. C. & Trapani, J. A. Perforin and granzymes: function, dysfunction and human pathology. Nat. Rev. Immunol. 15, 388-400 (2015).
Wang H, Li Z Y, Liu Y, Persson J, Beyer I, Moller T, Koyuncu D, Drescher M R, Strauss R, Zhang X B, et al.: Desmoglein 2 is a receptor for adenovirus serotypes 3, 7, 11 and 14. Nat Med 2011, 17:96-104.
Warming S, Costantino N, Court D L, Jenkins N A, Copeland N G: Simple and highly efficient BAC recombineering using galK selection. Nucleic Acids Res 2005, 33:e36.
White, E., Sabbatini, P., Debbas, M., Wold, W. S. M., Kusher, D. I., and Gooding, L. (1992). The 19-kilodalton adenovirus E 1B transforming protein inhibits programmed cell death and prevents cytolysis by tumor necrosis factor alpha. Mol. Cell. Biol. 1992; 12: 2570-2580).

### Cited Patent Publications:

U.S. Pat. No. 9,428,567
US2019062395
WO2014170389
WO2016146894

1. An oncolytic adenoviral vector comprising a nucleic acid sequence encoding a variant interleukin 2 (vIL-2) polypeptide as a transgene.

2. The oncolytic viral vector according to claim 1, wherein said variant IL-2 exhibits reduced binding to receptor subunit IL-2Rα but retains the IL-2Rβ and IL-2Rγ binding activity or has improved IL-2Rβ and IL-2Rγ binding activity.

3. The oncolytic vector according to claim 2, wherein a backbone of the oncolytic adenoviral vector is an adenovirus serotype 5 (Ad5) or serotype 3 (Ad3) nucleic acid backbone or adenovirus serotype 5 (Ad5) backbone with the fiber knob of adenovirus serotype 3 (Ad3)

4. The oncolytic vector according to claim 3, wherein said nucleic acid sequence encoding a variant interleukin 2 (vIL-2) polypeptide is in the place of a deleted nucleic acid sequence in the E3 region of said oncolytic adenoviral vector.

5. The oncolytic vector according to claim 4, wherein the deletion of a nucleic acid sequence in the E3 region is a deletion of viral gp19k and 6.7k reading frames.

6. The oncolytic vector according to claim 1, wherein the vector comprises a 24 bp deletion (424) in the adenoviral E1 sequence of said oncolytic adenoviral vector.

7. The oncolytic vector according to claim 1, wherein the vector comprises an Ad5/3 fiber knob.

8. The oncolytic vector according to claim 1, wherein the vector comprises Ad5/3-E2F-d24 backbone.

9. The oncolytic vector according to claim 1, wherein the vector has the structure Ad5/3-E2F-d24-vIL-2.

10. The oncolytic vector according to claim 1, wherein said nucleic acid sequence encodes for the variant IL-2 polypeptide comprising the substitutions L80F, R81D, L85V, I86V and I92F, the positions defined as in SEQ ID NO:2.

11. The oncolytic vector according to claim 1, wherein the vector comprises nucleic acid sequence encoding a further transgene.

12. The oncolytic vector according to claim 11, wherein the further transgene is encoding a cytokine.

13. The oncolytic vector according to claim 12, wherein the cytokine is selected from the list consisting of: TNFalpha, interferon alpha, interferon beta, interferon gamma, complement C5 a, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5 (=RANTES), CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2.

14. The oncolytic vector according to claim 12, wherein the cytokine is TNFalpha.

15. A pharmaceutical composition comprising an oncolytic adenoviral vector according to claim 1 and at least one of the following:
physiologically acceptable carriers, buffers, excipients, adjuvants, additives, antiseptics, preservatives, filling, stabilising and/or thickening agents.

16.-20. (canceled)

21. A method of treating cancer or tumor in a subject, wherein a pharmaceutically effective amount of an oncolytic adenoviral vector according to claim 1.

22. The method according to claim 21, wherein the cancer or tumor is selected from a group consisting of nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheo-chromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, esophagus cancer, gall bladder cancer, head cancer, eye cancer, neck cancer, kidney cancer, Wilms' tumor, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

23. The method according to claim 21, wherein said vector is administered together with an adoptive cell therapeutic composition.

24. The method according to claim 23, wherein the administration(s) of an adoptive cell therapeutic composition and an oncolytic viral vector to a subject is (are) conducted simultaneously or consecutively, in any order.

25. The method according to claim 21 further comprising administration of concurrent or sequential radiotherapy, monoclonal antibodies, chemotherapy, small molecular inhibitors, hormonal therapy, adoptive cell therapy or other anti-cancer drugs or interventions to a subject.

26. (canceled)


SEQUENCE LISTING (4 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - DNA, 531 bp, Adenovirus
atggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatga

SEQ ID NO: 4 - DNA, 390 bp, Adenovirus
atggaggcttgggagtgtttggaagatttttctgctgtgcgtaacttgctggaacagctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatga